(19) United States
(12) Patent Application Publication
(10) Pub. No.: US 2026/0354773 A1
(43) Pub. Date: Nov. 12, 2026

(54) METHOD FOR PROCESSING CORRECTIONAL DATA USING WEARABLE SENSORS

(71) Applicant: HLFIP Holding, LLC, Atlanta, GA (US)

(72) Inventor: Jonathan D. Logan, St. Petersburg, FL (US)

(73) Assignee: HLFIP Holding, LLC, Atlanta, GA (US)

(21) Appl. No.: 19/744,514

(22) Filed: Jul. 16, 2026

Related U.S. Application Data
(63) Continuation-in-part of application No. 19/217,435, filed on May 23, 2025.
Application No. 19/217,435 is a continuation of application No. 17/580,462, filed on Jan. 20, 2022, now Pat. No. 12,310,762.
(60) Provisional application No. 63/139,778, filed on Jan. 20, 2021.

(51) Int. Cl. A61B 5/00

(52) U.S. Cl. CPC A61B 5/746; A61B 5/0022; A61B 5/681; A61B 5/7405; A61B 5/742; A61B 5/7465

(57) ABSTRACT

A computer-implemented method for facilitating communication between computing devices in a networked environment. The method comprises establishing bidirectional communication channels between first computing devices and second computing devices through a communication hub. The system receives requests from the second computing devices and routes data between the devices using stored device identifiers and connection states. The communication protocol supports multiple data types including audio streams, image data, and screen display information. The system manages connection groups to enable multiple second computing devices to communicate with a single first computing device simultaneously. Communication sessions include timeout mechanisms and connection state verification to maintain system reliability. The method provides a generalized framework for managing real-time data exchange between distributed computing devices in various networked environments where coordinated information sharing is required.

### CROSS-REFERENCE TO RELATED APPLICATIONS

This application is a continuation-in-part of U.S. Patent Application 19/217,435, filed May 23, 2025, titled “SMART WEARABLE DEVICE FOR TRACKING AND MONITORING INDIVIDUALS IN A CORRECTIONAL FACILITY” which is a continuation of U.S. Patent Application 17/580,462, filed Jan. 20, 2022, titled “SMART WEARABLE DEVICE FOR TRACKING AND MONITORING INDIVIDUALS IN A CORRECTIONAL FACILITY.” which claims the benefit of U.S. Provisional Application 63/139,778, filed Jan. 20, 2021, titled “SMART WEARABLE DEVICE FOR TRACKING AND MONITORING INDIVIDUALS IN A CORRECTIONAL FACILITY.” The contents of the above identified applications are incorporated herein by reference in their entirety.

### BACKGROUND

### FIELD OF THE ART

The present disclosure is related to the field of remote monitoring systems, and more particularly to computer-implemented methods and systems for establishing secure communication channels between wearable devices and remote computing devices to enable real-time environmental monitoring and data capture within controlled facility environments.

### Discussion of the State of the Art

Correctional facility administrators require comprehensive awareness of activities occurring within their facilities to maintain security and manage inmate populations effectively. The ability to gather real-time intelligence about specific locations and individuals within a facility directly impacts operational safety and administrative decision-making.

Current surveillance methods in correctional facilities rely predominantly on fixed infrastructure systems. These systems typically consist of wall-mounted cameras positioned at predetermined locations throughout the facility, supplemented by microphone arrays installed in common areas. Physical inspections by correctional officers provide additional monitoring coverage through scheduled rounds and random checks of housing units, recreation areas, and other facility spaces.

Fixed surveillance infrastructure presents several operational limitations. Camera installations create predictable monitoring patterns that individuals can identify and circumvent. The static positioning of cameras results in blind spots where activities remain unobserved. Areas between camera coverage zones, spaces blocked by architectural features, and locations where cameras cannot be practically installed due to structural or privacy considerations all represent gaps in surveillance coverage. The installation and maintenance of comprehensive fixed camera systems requires substantial capital investment in equipment, wiring infrastructure, and ongoing technical support.

Attempts to address these limitations through mobile monitoring solutions have encountered technical obstacles. Existing mobile device-based systems require manual intervention to establish connections between monitoring devices and central control systems. This manual process introduces delays between the decision to monitor a location and the actual commencement of surveillance. Connection establishment procedures in current systems lack automation and require technical knowledge from operators, reducing the pool of personnel capable of initiating monitoring sessions.

Current mobile monitoring implementations demonstrate poor connection reliability. These systems fail to maintain stable connections when network conditions fluctuate or when devices move between coverage areas. Connection failures result in monitoring interruptions that compromise intelligence gathering efforts. When connections drop, existing systems typically require complete re-initialization of the monitoring session, creating gaps in surveillance coverage during critical periods.

The technical architecture of available monitoring solutions limits their scalability. These systems cannot support multiple monitoring clients accessing the same device simultaneously, restricting the number of personnel who can observe a particular location or individual. This limitation forces facilities to choose between providing access to senior administrators or operational staff, but not both groups concurrently. The inability to distribute monitoring capabilities across multiple authorized users reduces the effectiveness of intelligence gathering operations.

Connection management in existing systems lacks sophistication in handling network timeouts and temporary connectivity issues. When network latency increases or packets are lost, current implementations terminate sessions abruptly without attempting recovery. This behavior results in frequent monitoring interruptions in facilities where network infrastructure may be stressed by environmental factors or high usage periods.

Available monitoring solutions alert monitored individuals to surveillance activities through observable indicators. Device screens illuminate when monitoring begins, LED indicators activate to show camera operation, or audio feedback signals microphone activation. These notifications enable individuals engaged in prohibited activities to alter their behavior when surveillance commences, eliminating the intelligence value of the monitoring effort. The overt nature of current monitoring technologies transforms them from intelligence gathering tools into deterrence mechanisms, which serves a different operational purpose than covert observation.

Real-time data streaming capabilities in existing systems suffer from technical deficiencies. Current implementations exhibit high latency between event occurrence and observation at monitoring stations. This delay prevents immediate response to developing situations and reduces the actionable value of gathered intelligence. Video streams frequently experience frame drops, compression artifacts, and synchronization issues with audio channels. These quality degradations impair the ability of monitoring personnel to accurately assess situations and make informed decisions.

The absence of graceful degradation mechanisms in current systems creates operational disruptions. When optimal connection conditions cannot be maintained, existing solutions cease functioning entirely rather than adapting to available bandwidth or switching to lower-quality modes that would preserve basic monitoring capabilities. This all-or-nothing approach to connection management results in complete loss of surveillance coverage during network congestion or partial outages.

### SUMMARY

The present invention provides a computer-implemented system and method for remote monitoring of wearable computing devices within controlled facility environments. The system establishes bidirectional communication channels between wearable devices worn by monitored individuals and remote client computing devices operated by authorized personnel. Through a signaling protocol utilizing task completion sources and real-time communication hubs, the system enables activation of monitoring capabilities including audio capture, screen capture, and camera image acquisition.

The technical architecture employs an API entry point that interfaces with a communication hub to manage connection states, device registration, and data streaming. When a monitoring request is received, the system identifies the target wearable device from a database using device identifiers, verifies existing connection states, and establishes or utilizes communication channels. The system supports multiple simultaneous client connections to a single wearable device through group-based connection management, enabling real-time audio streaming with keep-alive mechanisms, on-demand screen captures, and camera image retrieval with automatic timeout handling.

The invention addresses technical challenges in remote device monitoring by implementing an API that receives monitoring requests and interfaces with a real-time communication hub. Upon receiving a monitoring request, the system queries a device database to identify the target wearable device, checks for existing connections using task completion sources, and either utilizes existing connections or initiates new connections through a watch service notification system. The communication hub implements group-based connection management using device-specific group identifiers, enabling multiple client computing devices to simultaneously receive monitoring data from a single wearable device. The audio monitoring subsystem implements a keep-alive mechanism requiring periodic refresh signals to maintain active streams, while screen capture and camera image retrieval utilize task completion sources that block until data is received or timeout thresholds are exceeded.

The system generates real-time transcripts of audio data using speech-to-text processing, creating searchable records of monitored communications. The system supports automated monitoring triggers based on conditions including incoming/outgoing communications, proximity detection between individuals, keyword analysis through watch firmware, biometric data changes, and location-based triggers. The architecture supports encrypted connections, historical location data retrieval, and real-time location display for multiple devices simultaneously. The system provides connection status feedback to client devices, enabling operators to verify monitoring session establishment before initiating data capture. The technical implementation provides remote monitoring capabilities with automatic connection management and failure handling through the integration of APIs, real-time communication protocols, and task-based asynchronous programming patterns.

In one aspect, the system architecture includes an integrated behavioral and biometric tracking pipeline. The system executes continuous covert audio and video streaming from the target wearable device, routing the parallel streams through a server-side analytics suite. The audio stream is processed concurrently by the transcription engine to produce real-time text logs, and a keyword tracking engine that parses the text logs against a dynamic database of flagged terminology to trigger automated administrative alerts. Concurrently, a voice biometrics engine extracts unique acoustic features from the audio stream to generate a biometric voiceprint, verifying the identity of the speaker against a stored baseline template. The system compiles the raw video streams, audio recordings, text transcripts, verified biometric sessions, and tracked keyword events into a centralized database repository structured as an objective inmate profile associated uniquely with the individual's inmate identifier.

### BRIEF DESCRIPTION OF THE DRAWINGS

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1 illustrates a network architecture for Method for Processing Correctional Data Using Wearable Sensors in accordance with an exemplary embodiment of the invention.

FIG. 2 illustrates a system architecture for Method for Processing Correctional Data Using Wearable Sensors in accordance with an exemplary embodiment of the present invention.

FIG. 3 illustrates an exemplary computer implemented process for Method for Processing Correctional Data Using Wearable Sensors according to one embodiment of the invention.

FIG. 4 illustrates one embodiment of the computing architecture that supports an embodiment of the inventive disclosure.

FIG. 5 illustrates components of a system architecture that supports an embodiment of the inventive disclosure.

FIG. 6 illustrates components of a computing device that supports an embodiment of the inventive disclosure.

FIG. 7 illustrates components of a computing device that supports an embodiment of the inventive disclosure.

### DETAILED DESCRIPTION

One or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural, logical, software, electrical and other changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible embodiments and in order to more fully illustrate one or more embodiments. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, method, or algorithm is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various embodiments in which, for example, functions may be executed out of order from that shown or discussed, including substantially concurrently or in reverse order, depending on the functionality involved, as would be understood by those having ordinary skill in the art.

The detailed description set forth herein in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

### Network Architecture

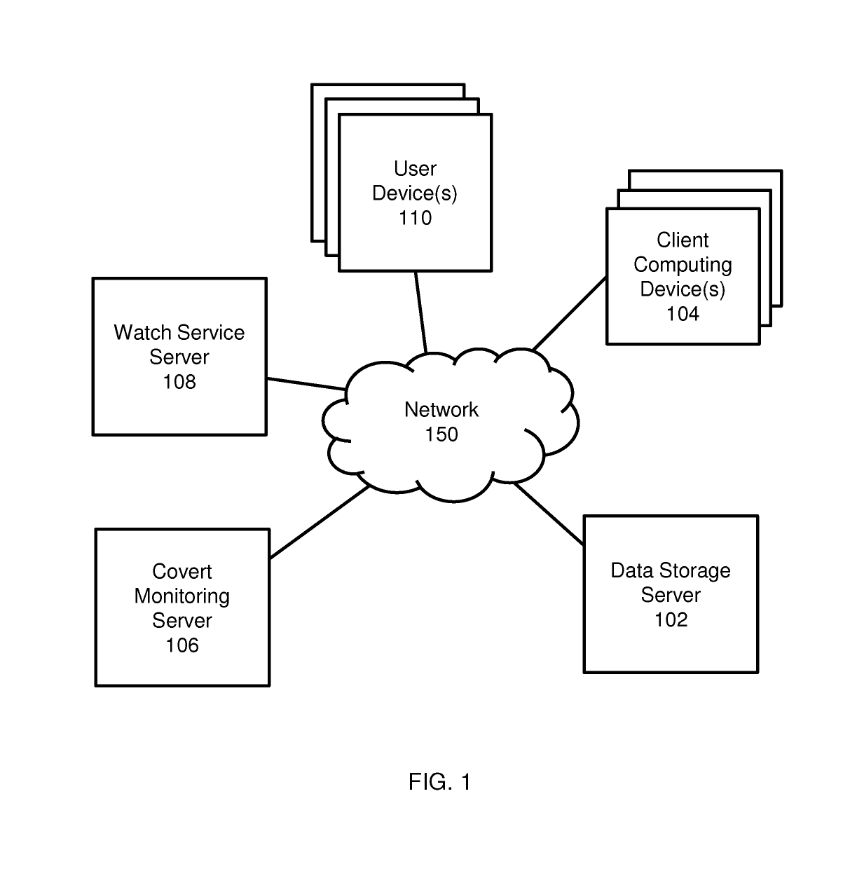

FIG. 1 illustrates an exemplary embodiment of a covert monitoring system architecture for wearable devices in a facility environment. The network diagram includes: a wearable user device 110, client computing device(s) 104, a covert monitoring server 106, a watch service server 108, a data storage server 102, and network 150. The various components described herein are exemplary and for illustration purposes only and any combination or subcombination of the various components may be used as would be apparent to one of ordinary skill in the art. The system may be reorganized or consolidated, as understood by a person of ordinary skill in the art, to perform the same tasks on one or more other servers or computing devices without departing from the scope of the invention.

At a high level, the system comprises a user device(s) 110 configured for deployment within a facility environment (e.g. a correctional facility) to enable remote monitoring capabilities of individuals associated with (e.g. wearing) the device. The user device 110 may take the form of a wearable user device such as a smartwatch or similar arm-worn computing platform that integrates multiple hardware components and specialized firmware to facilitate covert monitoring operations.

The user device(s) 110 functions to capture and transmit various forms of data from the wearer’s immediate environment while maintaining operational transparency to the wearer. The user device(s) 110 receives activation signals from remote monitoring systems, establishes real-time communication channels, and streams multiple data types including audio feeds, visual display information, and optionally photographic imagery. The user device(s) 110 operates without providing perceptible indicators to the wearer regarding active monitoring sessions, thereby preserving the covert nature of surveillance activities within the correctional facility.

The user device(s) 110 operates through coordinated interaction of its hardware and firmware components. The processor executes firmware instructions stored in memory to manage monitoring operations. Upon receiving monitoring activation signals through the wireless communication circuitry, the firmware initiates a SignalR connection to establish a persistent, bidirectional communication channel with the monitoring hub. The microphone component captures ambient audio which the firmware encodes and transmits as a continuous audio stream. The display component enables screen capture functionality, allowing the firmware to periodically capture and transmit the current display contents. When equipped with a camera, the user device 110 can acquire and transmit photographic images of the surrounding environment. The firmware manages these operations while suppressing standard user interface elements that would typically indicate active recording or transmission, such as notification icons, LED indicators, or audio feedback.

Alternative implementations of user device(s) 110 include deployment as a fitness tracker with reduced display capabilities but extended battery life, an ankle-mounted monitoring device incorporating similar sensing and communication components, or a pendant-style wearable device worn around the neck. The user device(s) 110 may alternatively use Bluetooth Low Energy for initial connection establishment before transitioning to Wi-Fi for data transmission, implement WebSocket protocols instead of SignalR for real-time communication, or employ MQTT messaging protocols for lightweight data transmission. The audio capture functionality may alternatively be implemented using multiple microphones for directional audio recording or bone conduction sensors for enhanced voice capture. Screen capture alternatives include transmitting only changed screen regions to reduce bandwidth consumption or implementing optical character recognition to transmit text-only representations of display content. The optional camera component may alternatively be implemented as an infrared camera for low-light operation or as a 360-degree camera for panoramic image capture.

User device(s) 110 include, generally, a computer or computing device including functionality for communicating (e.g., remotely) over a network 150. User device(s) 110 may comprise wearable user device(s). Data may be collected from user devices 110, and data requests may be initiated from each user device 110. User device(s) 110 may be a server, a desktop computer, a laptop computer, personal digital assistant (PDA), an in- or out-of-car navigation system, a smart phone or other cellular or mobile phone, or mobile gaming device, among other suitable computing devices. User devices 110 may execute one or more applications, such as a web browser (e.g., Microsoft Windows Internet Explorer, Mozilla Firefox, Apple Safari, Google Chrome, and Opera, etc.), or a dedicated application to submit user data, or to make prediction queries over a network 150.

In particular embodiments, each user device 110 may be an electronic device including hardware, software, or embedded logic components or a combination of two or more such components and capable of carrying out the appropriate functions implemented or supported by the user device 110. For example and without limitation, a user device 110 may be a desktop computer system, a notebook computer system, a netbook computer system, a handheld electronic device, or a mobile telephone. The present disclosure contemplates any user device 110. A user device 110 may enable a network user at the user device 110 to access network 150. A user device 110 may enable its user to communicate with other users at other user devices 110.

A user device 110 may have a web browser, such as MICROSOFT INTERNET EXPLORER, GOOGLE CHROME or MOZILLA FIREFOX, and may have one or more add-ons, plug-ins, or other extensions, such as TOOLBAR or YAHOO TOOLBAR. A user device 110 may enable a user to enter a Uniform Resource Locator (URL) or other address directing the web browser to a server, and the web browser may generate a Hyper Text Transfer Protocol (HTTP) request and communicate the HTTP request to server. The server may accept the HTTP request and communicate to the user device 110 one or more Hyper Text Markup Language (HTML) files responsive to the HTTP request. The user device 110 may render a web page based on the HTML files from server for presentation to the user. The present disclosure contemplates any suitable web page files. As an example and not by way of limitation, web pages may render from HTML files, Extensible Hyper Text Markup Language (XHTML) files, or Extensible Markup Language (XML) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a web page encompasses one or more corresponding web page files (which a browser may use to render the web page) and vice versa, where appropriate.

The user device 110 may also include an application that is loaded onto the user device 110. The application obtains data from the network 150 and displays it to the user within the application interface.

Exemplary user devices are illustrated in some of the subsequent figures provided herein. This disclosure contemplates any suitable number of user devices, including computing systems taking any suitable physical form. As example and not by way of limitation, computing systems may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, or a combination of two or more of these. Where appropriate, the computing system may include one or more computer systems; be unitary or distributed; span multiple locations; span multiple machines; or reside in a cloud, which may include one or more cloud components in one or more networks. Where appropriate, one or more computing systems may perform without substantial spatial or temporal limitation one or more steps of one or more methods described or illustrated herein. As an example, and not by way of limitation, one or more computing systems may perform in real time or in batch mode one or more steps of one or more methods described or illustrated herein. One or more computing system may perform at different times or at different locations one or more steps of one or more methods described or illustrated herein, where appropriate.

At a high level client computing device(s) 104 serve as an interface terminal for authorized facility personnel to interact with a monitoring system. The client computing device 104 functions as a control and observation station that enables personnel to initiate monitoring sessions and receive various forms of monitoring data from remote wearable devices. Client computing device(s) 104 may comprise the same or similar device as user device(s) 110 described above.

The client computing device 104 operates to establish communication channels with wearable devices, transmit monitoring commands, and receive multiple data streams including audio, visual imagery, and screen captures. The device 104 provides authorized personnel with the capability to monitor subjects wearing connected devices through a unified interface that consolidates different monitoring modalities. The client computing device 104 facilitates both individual monitoring sessions and group-based monitoring scenarios where multiple authorized users can observe the same wearable device simultaneously.

The client computing device 104 functions through an integrated architecture comprising hardware and software components. The processor executes client application software that manages communication protocols and data rendering. The network interface enables the client computing device 104 to transmit monitoring requests through API calls to initiate monitoring sessions. The client computing device 104 establishes persistent connections to a monitoring hub using SignalR technology, which enables bidirectional communication for real-time data streaming. Upon establishing these connections, the client computing device 104 receives audio streams that are processed and rendered through the device’s audio output components. The display component presents captured screen images and camera feeds to the operator. The client application software coordinates these various data streams and manages the user interface elements that allow operators to control monitoring parameters. When multiple client computing devices 104 connect to monitor a single wearable device, the monitoring hub manages these connections through group-based connection protocols that ensure synchronized data delivery to all connected clients.

Alternative implementations of the client computing device 104 include mobile computing devices such as tablets or smartphones equipped with corresponding mobile application software. The client computing device 104 can alternatively be implemented as a web-based terminal accessing monitoring functions through a web browser interface rather than dedicated client software. The SignalR connections can be replaced with WebSocket connections or long-polling mechanisms for maintaining persistent communication channels. Instead of API calls, the client computing device 104 can utilize message queue protocols or publish-subscribe messaging patterns for transmitting monitoring requests. The audio streaming functionality can be implemented using WebRTC protocols or proprietary streaming protocols. Rather than displaying all monitoring data on a single device, the client computing device 104 can be configured as a thin client that offloads processing to cloud-based servers, with the device primarily serving as a display terminal. The group-based hub connections can alternatively be implemented through peer-to-peer networking architectures or multicast protocols for distributing monitoring data to multiple clients.

At a high level, covert monitoring server 106 comprises a centralized computing infrastructure that coordinates monitoring activities between user devices (e.g. wearables) and client devices through network-based communication protocols. The covert monitoring server 106 functions as an intermediary hub that facilitates bidirectional data flow while maintaining persistent connections and managing device states across distributed monitoring sessions.

The covert monitoring server 106 enables real-time monitoring operations by receiving data streams from wearable devices, processing and routing this information to authorized client devices, and maintaining synchronized connection states between all participating devices. The server 106 accepts incoming connections from both wearable devices and client devices, authenticates these connections, establishes monitoring sessions, and continuously transfers monitoring data including audio streams, location information, sensor readings, and device status updates between source and destination endpoints while maintaining session integrity and connection reliability.

The covert monitoring server 106 operates through a multi-layered architecture comprising REST API endpoints for device registration and command processing, and a SignalR communication hub for persistent bidirectional connections. When a wearable device connects to the server 106, the server authenticates the device, registers it in the device database, and assigns it to a communication group. Client devices similarly connect and authenticate with the server 106, which then enables them to join monitoring sessions for authorized wearable devices. The server 106 maintains task completion sources for each active connection, allowing asynchronous handling of connection events and data transfers. During monitoring operations, the server 106 receives data packets from wearable devices through the SignalR hub, processes these packets to verify integrity and format, and forwards them to all connected client devices within the same monitoring group. The server 106 implements timeout mechanisms that detect disconnected devices and can automatically attempt reconnection or notify other participants of connection status changes.

Alternative implementations of the covert monitoring server 106 include utilizing WebSocket servers instead of SignalR for real-time communication, employing message queue systems such as RabbitMQ or Apache Kafka for data routing between devices, implementing the server using Node.js with Socket.io for cross-platform compatibility, deploying the server as containerized microservices using Docker or Kubernetes for scalability, utilizing GraphQL instead of REST API for more flexible query capabilities, implementing the server using gRPC for efficient binary protocol communication, employing peer-to-peer architectures where devices communicate directly after initial server-mediated discovery, using MQTT protocol servers for lightweight publish-subscribe messaging, implementing the server on serverless platforms using AWS Lambda or Azure Functions for automatic scaling, or utilizing WebRTC signaling servers that facilitate direct peer-to-peer connections after initial handshaking.

At a high level watch service server 108 comprises a server infrastructure that functions as an intermediary communication hub between monitoring systems and wearable devices deployed throughout a facility. The watch service server 108 operates as a centralized management system that coordinates the establishment of real-time communication channels between various system components and maintains operational oversight of connected wearable devices.

The watch service server 108 performs multiple functions within the monitoring ecosystem. The server 108 receives and processes connection initiation requests originating from covert monitoring servers within the network architecture. Upon receiving such requests, the watch service server 108 identifies target wearable devices based on parameters contained within the initiation requests and transmits notification signals to these specific devices. These notification signals contain instructions that direct the wearable devices to establish SignalR connections, which facilitate real-time bidirectional communication for monitoring sessions. Additionally, the watch service server 108 maintains a comprehensive database of device status information, including connection states, device availability, and operational parameters for each wearable device within the fleet.

The watch service server 108 operates through a multi-layered communication protocol. When a covert monitoring server requires activation of monitoring capabilities on a particular wearable device, it transmits a connection initiation request to the watch service server 108. The server 108 processes this request by querying its device status database to verify the availability and operational status of the target device. Upon confirmation, the watch service server 108 generates and transmits a notification signal containing connection parameters and authentication credentials to the target wearable device. The wearable device, upon receiving this notification, initiates a SignalR connection back to the designated monitoring endpoint. The watch service server 108 continues to monitor the connection status and can relay additional commands or configuration updates as required during the monitoring session.

Alternative implementations of the watch service server 108 include deployment as a distributed server cluster rather than a single server instance, enabling load balancing and redundancy across multiple server nodes. The communication protocol between the watch service server 108 and wearable devices could utilize WebSocket connections, MQTT (Message Queuing Telemetry Transport) protocol, or long-polling HTTP connections instead of SignalR. The notification mechanism could employ push notifications through platform-specific services such as Firebase Cloud Messaging or Apple Push Notification Service. The device status information could be maintained in a distributed database system or cached in a Redis cluster rather than a traditional database. The server 108 could be implemented as a containerized microservice running on orchestration platforms such as Kubernetes, or as a serverless function utilizing cloud computing services. The communication pathway could incorporate message queuing systems such as RabbitMQ or Apache Kafka to handle asynchronous message delivery between system components.

At a high level data storage server 102 comprises a computing system configured with persistent storage capabilities for archiving various types of monitoring data collected from monitored devices and systems. The data storage server 102 functions as a centralized repository that maintains multiple categories of information including audio recordings, screen captures, camera images, and generated transcripts, along with associated metadata and relational data structures that enable efficient retrieval and management of stored information.

The data storage server 102 operates to receive, process, and store monitoring data transmitted from various sources within the monitoring system. When monitoring data is received, the data storage server 102 categorizes and indexes the information according to predetermined schemas and storage protocols. The data storage server 102 maintains a device database that contains mappings between inmate identifiers and their corresponding wearable device identifiers, along with connection parameters required for establishing and maintaining communication links with the wearable devices. The data storage server 102 implements data retention policies that determine storage duration and archival procedures for different types of monitoring data.

The data storage server 102 works by implementing a multi-tiered storage architecture that organizes data based on access frequency and retention requirements. Real-time monitoring data is initially stored in high-speed storage arrays for immediate access, while older data is migrated to lower-tier storage systems according to defined policies. The data storage server 102 maintains relational database structures that link inmate identifiers to device identifiers through unique key relationships, enabling rapid lookup and retrieval of device-specific information. Connection parameters stored within the database include network addresses, authentication credentials, communication protocols, and device-specific configuration settings. The data storage server 102 continuously logs location data received from monitored devices, creating temporal records that track device movements and positions over time. Session logs are generated and stored for each monitoring session, capturing session initiation times, duration, participants, data types collected, and session termination details.

Alternative implementations of the data storage server 102 include distributed storage architectures where data is replicated across multiple geographic locations for redundancy and disaster recovery. Cloud-based storage solutions can be employed where monitoring data is stored in remote data centers managed by third-party providers with appropriate security certifications. Hybrid storage configurations can combine on-premises storage for sensitive data with cloud storage for less sensitive information. The device database can alternatively be implemented using non-relational database systems that store device mappings and parameters in document-oriented or key-value formats. Location data storage can utilize time-series databases optimized for temporal data rather than traditional relational database structures. Session logging can be implemented through dedicated log management systems that provide specialized indexing and search capabilities for audit trails.

Network cloud 150 generally represents a network or collection of networks (such as the Internet or a corporate intranet, or a combination of both) over which the various components illustrated in FIG. 1 (including other components that may be necessary to execute the system described herein, as would be readily understood to a person of ordinary skill in the art). In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 150 or a combination of two or more such networks 150. One or more links connect the systems and databases described herein to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable network 150, and any suitable link for connecting the various systems and databases described herein.

The network 150 connects the various systems and computing devices described or referenced herein. In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 421 or a combination of two or more such networks 150. The present disclosure contemplates any suitable network 150.

One or more links couple one or more systems, engines or devices to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable links coupling one or more systems, engines or devices to the network 150.

In particular embodiments, each system or engine may be a unitary server or may be a distributed server spanning multiple computers or multiple datacenters. Systems, engines, or modules may be of various types, such as, for example and without limitation, web server, news server, mail server, message server, advertising server, file server, application server, exchange server, database server, or proxy server. In particular embodiments, each system, engine or module may include hardware, software, or embedded logic components or a combination of two or more such components for carrying out the appropriate functionalities implemented or supported by their respective servers. For example, a web server is generally capable of hosting websites containing web pages or particular elements of web pages. More specifically, a web server may host HTML files or other file types, or may dynamically create or constitute files upon a request, and communicate them to client/user devices or other devices in response to HTTP or other requests from client devices or other devices. A mail server is generally capable of providing electronic mail services to various client devices or other devices. A database server is generally capable of providing an interface for managing data stored in one or more data stores.

In particular embodiments, one or more data storages may be communicatively linked to one or more servers via one or more links. In particular embodiments, data storages may be used to store various types of information. In particular embodiments, the information stored in data storages may be organized according to specific data structures. In particular embodiments, each data storage may be a relational database. Particular embodiments may provide interfaces that enable servers or clients to manage, e.g., retrieve, modify, add, or delete, the information stored in data storage.

The system may also contain other subsystems and databases, which are not illustrated in FIG. 1, but would be readily apparent to a person of ordinary skill in the art. For example, the system may include databases for storing data, storing features, storing outcomes (training sets), and storing models. Other databases and systems may be added or subtracted, as would be readily understood by a person of ordinary skill in the art, without departing from the scope of the invention.

### System Architecture

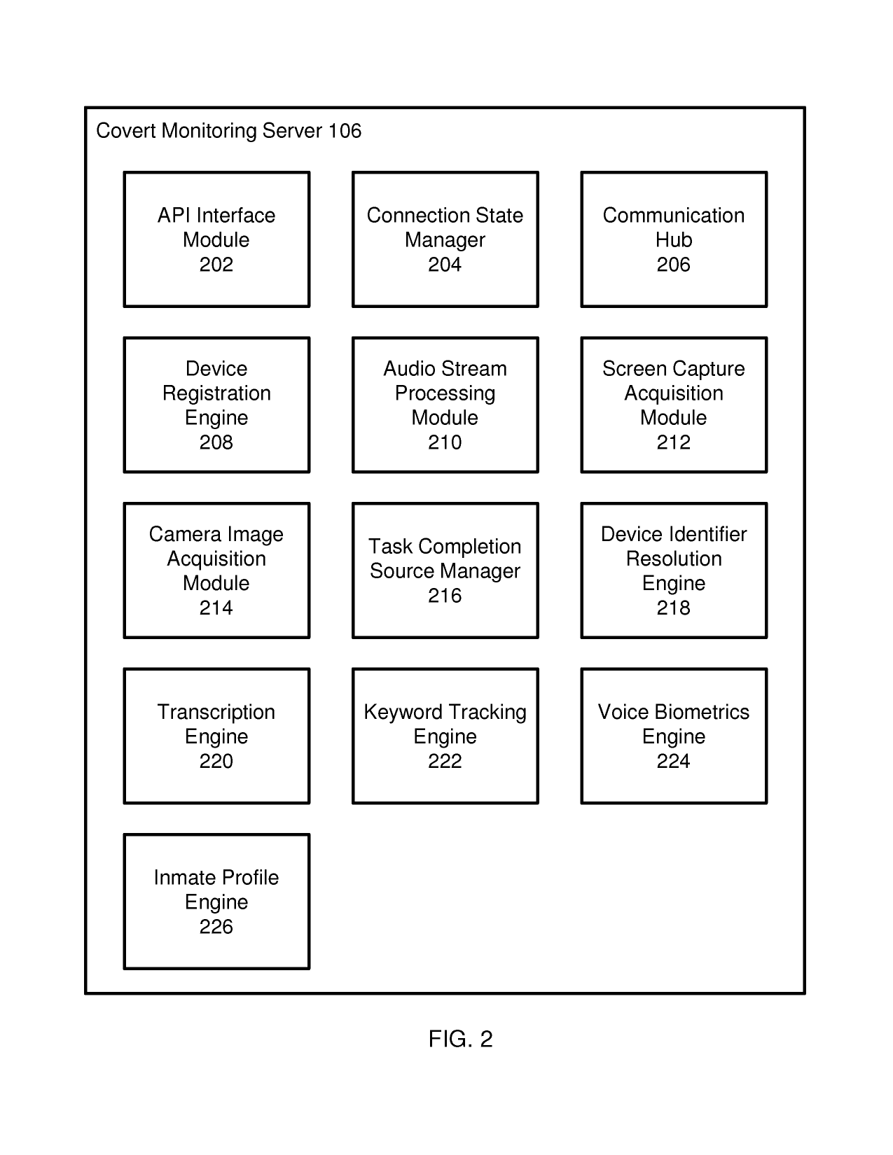

FIG. 2 illustrates an exemplary embodiment of the internal architecture of the covert monitoring server 106, showing the software modules and engines that execute the monitoring processes. The system includes: API interface module 202, connection state manager 204, communication hub 206, device registration engine 208, audio stream processing module 210, screen capture acquisition module 212, camera image acquisition module 214, task completion source manager 216, device identifier resolution engine 218, transcription engine 220, keyword tracking engine 222, voice biometrics engine 224, and inmate profile manager 226. The various components described herein are exemplary and for illustration purposes only and any combination or subcombination of the various components may be used as would be apparent to one of ordinary skill in the art. Other systems, interfaces, modules, engines, databases, and the like, may be used, as would be readily understood by a person of ordinary skill in the art, without departing from the scope of the invention. Any system, interface, module, engine, database, and the like may be divided into a plurality of such elements for achieving the same function without departing from the scope of the invention. Any system, interface, module, engine, database, and the like may be combined or consolidated into fewer of such elements for achieving the same function without departing from the scope of the invention. All functions of the components discussed herein may be initiated manually or may be automatically initiated when the criteria necessary to trigger action have been met.

At a high level, API interface module 202 serves as an entry point for client applications to interact with the monitoring system. The module may implement RESTful architectural principles to facilitate communication between client applications and the monitoring infrastructure through standardized HTTP-based protocols.

The API interface module 202 functions to receive and process monitoring requests from client applications seeking to establish monitoring sessions with target devices. The module exposes an OpenConnection endpoint that accepts HTTP requests containing monitoring parameters. Upon receiving a request, 202 performs authentication and authorization operations to verify that the requesting client possesses appropriate credentials to access the monitoring service. The module extracts target device identifiers from the incoming request payload and utilizes these identifiers to coordinate with other system components for connection establishment.

In operation, API interface module 202 receives an HTTP request at the OpenConnection endpoint, which may be formatted as a POST request containing JSON-encoded data in the request body. The module parses the request to extract authentication credentials, which may include API keys, OAuth tokens, or other authentication mechanisms. Following credential validation, API interface module 202 extracts the target device identifier from the request payload. The module then interfaces with a connection state manager component to determine whether an existing connection to the specified target device is already established. If no existing connection is found, API interface module 202 initiates a new connection establishment process. Upon successful connection establishment or identification of an existing connection, API interface module 202 generates a response containing a group identifier that corresponds to a SignalR hub connection. This group identifier enables the client application to subscribe to real-time updates through the SignalR communication framework.

Alternative implementations of API interface module 202 may employ different API architectural styles, such as GraphQL interfaces that allow clients to specify precise data requirements in their queries. Another alternative involves implementing a gRPC-based interface that utilizes Protocol Buffers for efficient binary serialization and supports bidirectional streaming capabilities. The module may alternatively be implemented using WebSocket connections directly, bypassing the REST paradigm entirely for scenarios requiring persistent connections from the initial handshake. Additionally, API interface module 202 may be configured to support multiple authentication schemes simultaneously, including certificate-based authentication, SAML assertions, or custom token formats specific to the monitoring system’s security requirements. Furthermore, monitoring requests may be automatically initiated via the user device. For example, the user device may be configured to listen for various keywords, sounds, sudden environmental volume changes or other trigger criteria indicating the user device should initiate monitoring and send such monitoring request to API interface module 202. Alternatively, the user device may automatically triggering recording of one or more forms of data in response to a trigger and store the data locally until a time when the monitoring data can be communicated to covert monitoring server.

At a high level, connection state manager 204 comprises a software module that monitors and maintains the operational state of communication links between remote wearable devices and a centralized monitoring hub. The connection state manager 204 operates as an intermediary layer that abstracts connection management complexity from other system components while providing real-time visibility into the status of established communication channels.

The connection state manager 204 performs multiple functions within the monitoring system architecture. The module maintains a registry of active SignalR connections, tracking each connection’s current state, associated device identifiers, and temporal characteristics. The connection state manager 204 processes connection lifecycle events, including establishment, maintenance, and termination of communication channels. The module implements timeout mechanisms that detect and respond to unresponsive connections, enabling the system to distinguish between temporary network interruptions and permanent connection failures. Additionally, 204 provides an interface through which other system components can query connection status and request connection establishment or termination operations.

In operation, the connection state manager 204 maintains several data structures to track connection state information. A connection registry stores active connection objects indexed by device identifiers, enabling rapid lookup of existing connections. Task completion sources are associated with pending connection requests, allowing asynchronous connection establishment while maintaining request context. The module implements a timeout monitoring mechanism that periodically evaluates connection timestamps against configurable threshold values. When a connection request is received, 204 first queries its internal registry to determine if an active connection exists for the specified device. If an existing connection is found and remains within acceptable timeout parameters, the module returns a reference to the existing connection. If no suitable connection exists, 204 initiates a new connection establishment procedure, creates a corresponding task completion source, and monitors the connection attempt for successful completion or timeout expiration.

Alternative implementations of the connection state manager 204 may employ different architectural patterns and technologies. Instead of SignalR, the module could utilize WebSocket connections directly, implement long-polling mechanisms, or employ message queuing protocols such as MQTT or AMQP. The connection registry could be implemented using distributed caching systems to enable horizontal scaling across multiple monitoring hub instances. Rather than maintaining task completion sources, the module could implement a callback-based architecture or utilize reactive programming patterns with observable streams. The timeout mechanism could be replaced with heartbeat protocols where connected devices periodically transmit keep-alive messages. Additionally, the connection state manager 204 could be implemented as a microservice with its own dedicated process space, communicating with other system components through inter-process communication mechanisms or RESTful APIs.

At a high level, communication hub 206 comprises a real-time communication infrastructure that facilitates bidirectional data exchange between wearable devices and client applications. The communication hub 206 operates as an intermediary layer that manages persistent connections and enables push-based communication patterns for streaming data from wearable devices to one or more connected clients. The communication hub 206 may comprise a SignalR-based communication hub.

The communication hub 206 provides functionality for establishing and maintaining real-time connections between disparate system components. In one embodiment, 206 enables wearable devices to transmit audio streams, screen captures, and camera images to client applications without requiring continuous polling. The hub 206 further provides connection management capabilities that allow multiple client applications to simultaneously receive data streams from a single wearable device source.

In operation, the communication hub 206 implements a group-based connection architecture wherein each wearable device is associated with a unique group identifier following a predetermined format, such as “WATCH_deviceID”. When a client application initiates a connection to communication hub 206, it can register interest in receiving data from a specific user device by joining the corresponding group. The communication hub 206 maintains a registry of active connections and their associated group memberships, enabling targeted message delivery to specific client subsets.

The communication hub 206 comprises several processes that facilitate device interaction. A register process allows wearable devices to establish their presence within the system and create their associated communication group. A start audio monitor process initiates audio data streaming from a specified device. An audio stream process handles the transmission of audio data packets from the wearable device to subscribed clients. A get screen capture process enables clients to request and receive screen capture data from a connected wearable device. A get camera image process provides similar functionality for camera image acquisition.

The communication hub 206 may employ WebSocket connections as its primary transport mechanism when available, with automatic fallback to alternative transport protocols such as Server-Sent Events or long polling when WebSocket connections cannot be established. The communication hub 206 implements automatic reconnection logic to handle temporary network disruptions and maintains connection state information to ensure message delivery reliability.

Alternative implementations of the real-time communication functionality provided by 206 may include WebRTC-based peer-to-peer communication systems, MQTT message brokers, Apache Kafka streaming platforms, or custom TCP/IP socket implementations. REST-based polling mechanisms could be employed as an alternative, though with reduced real-time performance characteristics. GraphQL subscriptions or gRPC streaming could also provide similar bidirectional communication capabilities. Additionally, cloud-based messaging services such as Azure Service Bus, AWS IoT Core, or Google Cloud Pub/Sub could serve as alternatives for managing device-to-client communication patterns.

At a high level, device registration engine 208 comprises a software component that facilitates the establishment of connections between wearable devices and a centralized communication infrastructure. The device registration engine 208 operates as an intermediary processing layer that manages incoming connection requests and orchestrates the integration of new devices into the system’s communication framework.

The device registration engine 208 functions to receive and process registration requests transmitted from wearable devices seeking to establish communication channels with the SignalR hub infrastructure. Upon receipt of a registration request, device registration engine 208 extracts connection parameters including device identifiers and group designation information. The engine validates the registration parameters and initiates the connection establishment protocol. Following successful validation, device registration engine 208 integrates the requesting device into the designated communication group, thereby enabling bidirectional data exchange between the device and other system components. The engine further maintains synchronization with pending operations by signaling task completion mechanisms and updating system state information to reflect the newly established connection.

In operation, device registration engine 208 implements a multi-stage registration workflow. When a wearable device initiates a registration call, the call includes a group name parameter that specifies the intended communication group for the device. The device registration engine 208 parses this registration call to extract the group name and connection identifier. The engine then invokes group management functions to add the device connection to the specified group within the SignalR hub architecture. Concurrently, device registration engine 208 queries for any pending task completion sources that may be awaiting the establishment of this particular connection. Upon identifying such pending tasks, the engine signals these completion sources to indicate that the requested connection has been successfully established. Additionally, device registration engine 208 interfaces with a connection state manager component to update the system’s connection registry, marking the new connection as active and available for communication operations.

Alternative implementations of the device registration engine 208 may employ different communication protocols beyond SignalR, such as WebSocket-based frameworks, MQTT brokers, or custom TCP/IP socket implementations. The registration workflow may alternatively utilize RESTful API endpoints for device registration rather than real-time hub connections. In some embodiments, device registration engine 208 may implement a queuing mechanism to handle concurrent registration requests, employing message queue systems such as RabbitMQ or Apache Kafka. The group management functionality may be replaced with topic-based subscription models or channel-based architectures. Furthermore, the connection state management aspect may be implemented using distributed caching systems like Redis or in-memory data grids to maintain connection status across multiple server instances in scaled deployments.

At a high level, audio stream processing module 210 comprises a software module configured to facilitate real-time audio data transmission between wearable devices and connected client systems. The audio stream processing module 210 operates as an intermediary component that receives, processes, and distributes audio stream data across a networked environment, enabling multiple client systems to simultaneously monitor audio feeds from wearable devices.

The audio stream processing module 210 performs several functions within the system architecture. The audio stream processing module 210 establishes and maintains active audio streaming sessions between wearable devices and client systems through implementation of a keep-alive protocol. The audio stream processing module 210 receives audio stream packets transmitted from wearable devices, processes these packets to extract audio data, and distributes the processed audio data to appropriate client destinations. The module 210 manages group-based audio distribution, wherein audio data from a particular wearable device is transmitted to all client systems associated with that device’s designated group, enabling concurrent monitoring capabilities across multiple operator stations.

In operation, the audio stream processing module 210 implements a session management mechanism that utilizes periodic start audio monitor calls to maintain active audio streams. When a client system initiates an audio monitoring session, the module 210 registers the session and begins accepting audio stream packets from the associated wearable device. The module 210 monitors the timing of start audio monitor calls and terminates sessions that fail to receive timely keep-alive signals, thereby conserving system resources and maintaining session integrity. Upon receiving audio stream packets from a wearable device, the module 210 performs packet validation, extracts the audio payload, and identifies all client systems connected to the device’s assigned group. The module 210 then replicates and transmits the audio data to each identified client system, ensuring synchronized audio delivery across all monitoring stations.

Alternative implementations of the audio stream processing module 210 may employ different architectural approaches while maintaining similar functionality. One alternative embodiment may utilize a publish-subscribe messaging pattern, wherein wearable devices publish audio data to topic-based channels and client systems subscribe to relevant channels for receiving audio streams. Another alternative may implement a peer-to-peer audio distribution model, where the module 210 facilitates direct connections between wearable devices and client systems, reducing server-side processing requirements. An additional alternative may incorporate audio transcoding capabilities within the module 210, enabling dynamic conversion of audio formats based on client system capabilities or network conditions. Further alternatives may include implementation of adaptive bitrate streaming, wherein the module 210 adjusts audio quality based on available bandwidth, or incorporation of audio mixing capabilities for combining multiple audio streams from different wearable devices into composite feeds for client consumption.

At a high level, screen capture acquisition module 212 comprises a software subsystem that facilitates the acquisition of screen capture data from remote wearable devices in response to client application requests. The module serves as an intermediary between client applications and target wearable devices, managing the request-response lifecycle for screen capture operations.

The screen capture acquisition module 212 operates to provide real-time or near real-time screen capture functionality for wearable devices. When a client application requires a visual representation of the current display state of a wearable device, the screen capture acquisition module 212 receives a get screen capture request and orchestrates the capture process. The module returns compressed image data representing the wearable device’s screen content to the requesting client application, enabling remote monitoring, debugging, or user interface verification capabilities.

In operation, the screen capture acquisition module 212 implements a synchronous request-handling mechanism. Upon receiving a get screen capture request from a client application, the screen capture acquisition module 212 instantiates a task completion source object that serves as a synchronization primitive. The screen capture acquisition module 212 then formulates and transmits a capture request message to the designated target wearable device through an established communication channel. The screen capture acquisition module 212 enters a blocking state, awaiting a response from the wearable device containing the requested screen capture data. During this blocking period, the screen capture acquisition module 212 monitors for either the receipt of JPEG-encoded image data from the wearable device or the expiration of a predetermined timeout threshold. If the wearable device successfully captures and transmits the screen image data within the timeout period, the screen capture acquisition module 212 receives the JPEG data stream and returns it to the requesting client application. If the timeout threshold is exceeded before receiving a complete response, the screen capture acquisition module 212 may return an error indication or null response to the client application.

Alternative implementations of the screen capture acquisition functionality may employ various architectural and technical approaches. In one embodiment, the screen capture acquisition module 212 may utilize an asynchronous callback-based mechanism rather than blocking operations, wherein the client application provides a callback function that is invoked upon completion of the screen capture operation. Another embodiment may implement a polling-based approach where the screen capture acquisition module 212 returns a request identifier immediately to the client, and the client subsequently polls for completion status and retrieves the image data when available. The image data format may vary in different embodiments, with alternatives including PNG, BMP, WebP, or raw bitmap data instead of or in addition to JPEG encoding. Some embodiments may incorporate image compression negotiation, where the screen capture acquisition module 212 and wearable device dynamically select an optimal compression format based on network conditions, device capabilities, or client preferences. The communication protocol between screen capture acquisition module 212 and the wearable device may utilize REST APIs, WebSocket connections, gRPC, or proprietary binary protocols. Additionally, certain embodiments may implement caching mechanisms within screen capture acquisition module 212 to store recently captured screens, reducing redundant capture operations when multiple clients request the same screen state within a short time window.

At a high level, camera image acquisition module 214 comprises a software-based image acquisition system that facilitates camera-based photograph capture operations within a wearable device ecosystem. The camera image acquisition module 214 serves as an intermediary layer between client applications requesting photographic content and the physical camera hardware integrated within wearable devices.

The camera image acquisition camera image acquisition module 214 performs several functions within the overall system architecture. The camera image acquisition module 214 receives and processes image capture requests from client applications or other system components. Upon receiving such requests, the camera image acquisition module 214 formulates and transmits get camera image command packets to designated target wearable devices equipped with camera sensors. The camera image acquisition module 214 manages asynchronous communication patterns by implementing task completion source mechanisms that enable synchronous-style response handling despite the underlying asynchronous nature of device communications. When camera-equipped devices capture requested images, the camera image acquisition module 214 receives the resulting image data through established communication channels. The camera image acquisition module 214 subsequently delivers the acquired photographic data to the original requesting clients in appropriate formats. Additionally, the camera image acquisition module 214 implements timeout mechanisms to handle scenarios where target devices fail to respond within predetermined time intervals.

In operation, the camera image acquisition camera image acquisition module 214 employs a request-response communication pattern. When a client initiates an image capture request, the camera image acquisition module 214 creates a task completion source object that enables the calling thread to await the asynchronous operation’s completion. The camera image acquisition module 214 constructs a get camera image request packet containing relevant parameters such as image resolution specifications, compression settings, or camera selection indicators for devices with multiple cameras. This request packet is transmitted through the system’s communication infrastructure to the target wearable device. The camera image acquisition module 214 maintains a registry of pending requests indexed by unique identifiers, allowing correlation of responses with their originating requests. Upon receiving image data from the wearable device, the camera image acquisition module 214 extracts the photographic content, performs any required format conversions or data validation, and signals the task completion source to deliver the image to the waiting client. If the predetermined timeout period expires before receiving a response, the camera image acquisition module 214 cancels the pending operation and returns an appropriate error indication to the client.

Alternative implementations of the camera image acquisition functionality may be employed within the system. Instead of a dedicated camera image acquisition module 214, the image acquisition capabilities could be integrated directly into a broader device communication manager that handles multiple types of device interactions. The synchronous response handling mechanism utilizing task completion sources could be replaced with callback-based asynchronous patterns, where clients register callback functions to be invoked upon image receipt. Rather than implementing timeout handling within the camera image acquisition module 214, a separate watchdog service could monitor all pending device communications and trigger cancellations for expired operations. The get camera image request format could be substituted with a more generic command structure that supports various media capture types, including video or audio, through polymorphic request handling. Additionally, the camera image acquisition module 214 could implement a caching layer that stores recently captured images, enabling rapid response to repeated requests for the same photographic content without requiring additional device communications.

At a high level, task completion source manager 216 comprises an asynchronous operation coordination subsystem that manages task completion sources for connection establishment and data acquisition requests within the software system. The subsystem 216 facilitates non-blocking execution patterns by providing mechanisms to create, monitor, and signal completion states for pending operations, particularly those involving device communication that may experience variable response latencies.

The task completion source manager 216 operates to coordinate asynchronous workflows by instantiating task completion source objects that serve as synchronization primitives between requesting components and device response handlers. When a connection establishment or data acquisition request is initiated, task completion source manager 216 generates a corresponding task completion source that represents the pending operation’s future completion state. The subsystem task completion source manager 216 maintains references to these task completion sources and provides interfaces for both the signaling of completion events and the awaiting of such signals by dependent operations. Through this mechanism, task completion source manager 216 enables system components to initiate device communication requests and subsequently block or continue execution based on configurable waiting strategies.

In operation, task completion source manager 216 functions through a multi-component architecture comprising a task completion source factory, a tracking registry, and a timeout management component. When a request for asynchronous operation coordination is received, the task completion source factory within task completion source manager 216 creates a new task completion source instance configured with operation-specific parameters, including timeout thresholds. The tracking registry maintains associations between active operations and their corresponding task completion sources, enabling task completion source manager 216 to manage multiple concurrent asynchronous operations. The timeout management component monitors elapsed time for each pending operation and triggers timeout events when configured thresholds are exceeded. Upon receiving a device response, task completion source manager 216 locates the appropriate task completion source through the tracking registry and signals completion, allowing waiting operations to proceed. If a timeout occurs before a response is received, task completion source manager 216 signals the task completion source with a timeout indication, permitting graceful handling of non-responsive device scenarios.

Alternative implementations of task completion source manager 216 may employ callback-based coordination mechanisms wherein completion notifications are delivered through registered callback functions rather than task-based synchronization primitives. Another alternative embodiment may utilize promise/future patterns where task completion source manager 216 manages promise objects that encapsulate pending operation results. Event-driven architectures represent another alternative wherein task completion source manager 216 publishes completion events to an event bus for consumption by interested subscribers. Message queue-based implementations may also be employed where 216 deposits completion notifications into designated message queues for asynchronous processing. State machine-based alternatives may implement task completion source manager 216 as a finite state machine that transitions through defined states representing various stages of operation completion.

At a high level, device identifier resolution engine 218 comprises a software subsystem that facilitates the correlation between inmate identifiers and their associated wearable device identifiers within a monitoring system. The device identifier resolution engine 218 operates as an intermediary component that enables the system to establish monitoring sessions by resolving human-readable or system-assigned inmate identifiers into machine-readable device identifiers.

The device identifier resolution engine 218 performs automated mapping operations between inmate identifiers received through monitoring requests and corresponding wearable device identifiers maintained in a device database. When a monitoring request is initiated, 218 receives an inmate identifier as input, which may comprise various formats including alphanumeric strings, numerical codes, biometric identifiers, or other unique identifiers assigned to monitored individuals. The device identifier resolution engine 218 processes these incoming identifiers and executes database queries to retrieve associated device information, including but not limited to wearable device identifiers, group identifiers for batch processing, and hub connection parameters that facilitate communication with specific monitoring devices.

In operation, device identifier resolution engine 218 implements a multi-stage resolution process. Upon receiving a monitoring request containing an inmate identifier, the device identifier resolution engine 218 first validates the format and structure of the received identifier. The device identifier resolution engine 218 then constructs and executes one or more database queries against the device database, utilizing indexing structures and query optimization techniques to efficiently locate matching records. The resolution process performed by device identifier resolution engine 218 retrieves a comprehensive set of device-related parameters, including the unique wearable device identifier assigned to the monitored individual’s device, any group identifiers that may be used for collective monitoring operations, and hub connection parameters such as network addresses, communication protocols, authentication credentials, and encryption keys required for establishing secure monitoring sessions. The device identifier resolution engine 218 may implement caching mechanisms to store frequently accessed identifier mappings, thereby reducing database query overhead and improving response times for subsequent resolution requests.

Alternative implementations of device identifier resolution engine 218 may include distributed resolution architectures where identifier mapping operations are performed across multiple processing nodes to handle high-volume monitoring requests. Another embodiment may incorporate machine learning algorithms within device identifier resolution engine 218 to predict and pre-fetch device identifiers based on historical access patterns and monitoring schedules. The device identifier resolution engine 218 may alternatively implement a hierarchical resolution scheme where identifiers are resolved through multiple database tiers, with frequently accessed mappings stored in high-speed memory caches and less frequently accessed mappings maintained in persistent storage. Additionally, device identifier resolution engine 218 may be configured to support federated resolution across multiple device databases, enabling cross-facility or cross-jurisdictional monitoring capabilities. In certain embodiments, device identifier resolution engine 218 may implement real-time synchronization with external identity management systems to maintain current identifier mappings as monitored individuals are added, removed, or reassigned within the monitoring system.

At a high level, transcription engine 220 comprises a speech-to-text transcription engine that converts audio data from monitored communications into textual representations. The transcription engine 220 forms part of a communication monitoring system and operates to transform audio content into machine-readable and human-readable text formats for subsequent processing, storage, and analysis.

The speech-to-text transcription engine 220 functions to receive audio data streams from communication channels and generate corresponding text transcripts. The engine 220 processes various forms of audio input, including voice communications, recorded messages, and real-time audio feeds. The transcription engine 220 outputs textual data that represents the spoken content within the audio streams, enabling text-based searching, analysis, and archival of communication content that originates in audio format.

In operation, the speech-to-text transcription engine 220 receives digital audio data through an input interface that accepts audio streams in various formats and sampling rates. The engine 220 preprocesses the audio data by applying signal processing techniques such as noise reduction, normalization, and segmentation. The preprocessed audio undergoes feature extraction where acoustic characteristics are converted into numerical representations suitable for speech recognition processing. The engine 220 employs speech recognition algorithms that may include acoustic models, language models, and pronunciation dictionaries to decode the audio features into text sequences. The acoustic models map audio features to phonetic units, while language models provide context and grammatical structure to improve transcription accuracy. The engine 220 may utilize neural network architectures, such as recurrent neural networks or transformer-based models, to perform the speech-to-text conversion. The transcription process may operate in real-time streaming mode, where text is generated incrementally as audio is received, or in batch mode for processing stored audio files. The engine 220 includes post-processing components that format the raw transcription output, including punctuation insertion, capitalization, and speaker diarization when multiple speakers are present. The generated transcripts are transmitted through an output interface to client devices for real-time display or routed to data storage servers for archival and subsequent retrieval.

Alternative implementations of the speech-to-text functionality may be employed within the system architecture. Cloud-based speech recognition services may be utilized, where audio data is transmitted to remote servers that perform the transcription and return text results. Hybrid approaches may combine on-premises processing for sensitive communications with cloud services for standard transcriptions. The engine 220 may incorporate multiple speech recognition technologies operating in parallel, with result fusion techniques to improve accuracy. Speaker-adapted models may be implemented where the engine 220 customizes recognition parameters based on individual speaker characteristics. The system may employ phonetic transcription as an intermediate representation, particularly useful for languages with complex orthographies or for cross-lingual applications. Alternative acoustic modeling approaches include Hidden Markov Models, Deep Neural Networks, or end-to-end neural architectures that directly map audio to text without explicit phonetic modeling. The engine 220 may integrate with natural language processing components to enhance transcription quality through contextual understanding and domain-specific vocabulary models.

At a high level, keyword tracking engine 222 comprises an algorithmic text-parsing system that intercepts text data generated by transcription engine 220. The engine 222 operates to perform real-time natural language processing (NLP) search operations against a facility-configured dictionary of restricted terms, slang, or threat-indicative phrases. When a keyword match is identified within a streaming transcript, keyword tracking engine 222 appends a metadata flag to that specific timestamp of the recording, registers a keyword event log, and pushes an immediate alert token to client computing devices 104.

At a high level, voice biometrics engine 224 comprises an acoustic analytics subsystem configured to extract physiological and behavioral voice characteristics from audio stream packets. The voice biometrics engine 224 processes the audio data to isolate the voice print of the wearer from ambient noise, mapping features such as fundamental frequency, formant frequencies, and linear predictive coding coefficients. This runtime voiceprint is converted into a multi-dimensional biometric hash vector. Engine 224 compares this vector against a baseline biometric template stored in the inmate's profile to verify that the individual speaking is genuinely the assigned wearer of the user device 110, logging identity verification confidence scores throughout the session.

At a high level, inmate profile manager 226 comprises a database lifecycle application layer that structures, updates, and maintains consolidated inmate files within data storage server 102. Rather than isolated data logs, inmate profile manager 226 aggregates all surveillance outputs into a single object-oriented or relational inmate profile. The inmate profile manager 226 securely binds the inmate identifier to a dossier containing: historical baseline voice biometric templates, continuous audio/video recordings, synchronized textual transcripts, indexed keyword violation logs, and historical device event metrics, exposing a unified query interface for authorized clients.

### Computer Implemented Process

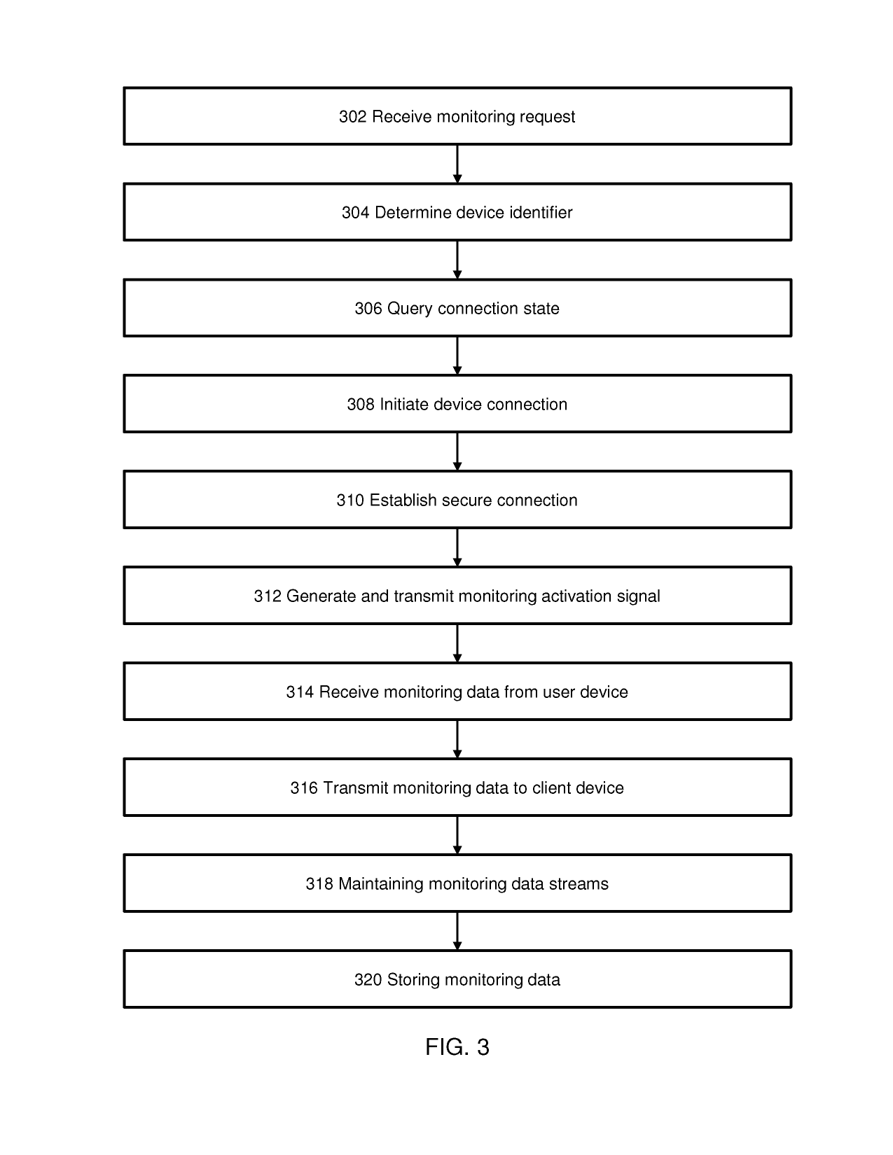

FIG. 3 illustrates an exemplary process flow diagram depicting a computer-implemented method for covertly monitoring the area around a user device within a facility, executed from the perspective of the covert monitoring server processor. The process includes the following steps: receiving a monitoring request from a client computing device (step 302), resolving the device identifier from the database based on the monitoring request (step 304), querying the connection state to determine existing connection status (step 306), initiating device connection via the watch service if no existing connection is found (step 308), establishing a secure bidirectional connection between the wearable device and the monitoring hub (step 310), generating and transmitting a monitoring activation signal to the wearable device (step 312), receiving monitoring data including audio streams and/or image data from the wearable device (step 314), transmitting the monitoring data to the client computing device (step 316), maintaining audio streams through keep-alive signal monitoring (step 318); storing monitoring data to the data storage server (step 320). The process steps described herein may be performed in association with a system such as that described in FIG. 1 and/or FIG. 2 above or in association with a different system. The process may comprise additional steps, fewer steps, and/or a different order of steps without departing from the scope of the invention as would be apparent to one of ordinary skill in the art.

At 302, the process comprises receiving a monitoring request from a client computing device via an API call to the REST API interface. The monitoring request includes an identifier associated with a target user incarcerated at a correctional facility. The server extracts the identifier and passes it to the device identifier resolution engine for processing.

The monitoring request received at 302 initiates the establishment of surveillance parameters for communications associated with an incarcerated individual within a correctional facility environment. The request enables authorized personnel, such as correctional officers, investigators, or administrative staff, to configure real-time or historical monitoring of electronic communications, including voice calls, video communications, electronic messages, and data transfers originating from or directed to the specified target user.

At 302, the REST API interface operates as a standardized communication endpoint that accepts HTTP-based requests from authenticated client computing devices. The API call comprises a structured data payload formatted according to predetermined specifications, such as JSON or XML format. The monitoring request payload contains the identifier, which may comprise an inmate identification number, a booking number, a system-generated unique identifier, a biometric hash value, or a combination thereof. The REST API interface implements authentication mechanisms to verify the requesting client’s authorization level before processing the monitoring request. Upon successful authentication, the server’s request handler parses the incoming API call to extract the identifier and any additional parameters, such as monitoring duration, communication types to monitor, or specific contact restrictions.

The extraction and processing at 302 involves the server’s request processing module parsing the API call’s data structure to isolate the identifier field. The server validates the identifier format against predetermined patterns using regular expression matching or format validation rules. Following validation, the server creates an internal request object containing the extracted identifier and routes this object to the device identifier resolution engine through an internal messaging queue or direct method invocation. The device identifier resolution engine maintains mappings between user identifiers and associated communication devices, accounts, or endpoints registered within the correctional facility’s communication system.

Alternative implementations for receiving the monitoring request at 302 include utilizing a SOAP-based web service interface instead of REST API, where the monitoring request arrives as an XML-formatted SOAP envelope containing the identifier within a structured message body. Another alternative involves implementing a GraphQL API endpoint that accepts query-based requests, allowing the client to specify precisely which monitoring parameters and identifier fields to include in the request. The server may alternatively receive monitoring requests through a message queue system, such as RabbitMQ or Apache Kafka, where client devices publish monitoring requests to a designated queue, and the server consumes these messages asynchronously. In another alternative, the monitoring request at 302 arrives via a WebSocket connection, enabling persistent bi-directional communication between the client and server for real-time monitoring updates. The identifier extraction process may alternatively utilize a dedicated parsing service or microservice that specializes in handling various identifier formats and validation rules, communicating with the main server through inter-process communication mechanisms or network protocols.

At 304, the process automatically queries the device database to resolve the inmate identifier to a corresponding wearable device identifier. This database query operation enables the system to establish the linkage between an inmate’s identification information and their assigned monitoring device, facilitating subsequent monitoring and communication operations.

At 304, the process comprises performing a database lookup operation that translates human-readable or system-assigned inmate identifiers into technical device identifiers that can be used for wireless communication and device management. The query retrieves multiple data elements including the wearable identifier, a group identifier formatted as WATCH_deviceID, and connection parameters such as communication protocols, frequency bands, encryption keys, and network addressing information required for establishing secure communication channels with the wearable device.

At 304, the process comprises receiving the inmate identifier as an input parameter, formulating a database query using structured query language (SQL) or similar database query protocols, and submitting the query to the device database management system. The database management system processes the query against indexed tables containing mappings between inmate identifiers and device identifiers. Upon locating the corresponding record, the system retrieves the complete device profile including the unique wearable identifier assigned during device provisioning, the group identifier that concatenates a device type prefix (WATCH) with the device ID using an underscore delimiter, and connection parameters stored as configuration data. These connection parameters may include wireless protocol specifications (such as Bluetooth Low Energy, Wi-Fi, or cellular network parameters), security credentials, communication timeouts, retry policies, and quality of service settings. The retrieved information is then returned to the requesting process for use in establishing the monitoring session.

At 304, alternative implementations may utilize different data storage and retrieval mechanisms. Instead of a relational database, the system may employ a key-value store where inmate identifiers serve as keys that directly map to device information objects. Another alternative involves using a distributed database system with replicated data across multiple nodes to ensure high availability and fault tolerance. The system may alternatively implement a caching layer that stores frequently accessed inmate-to-device mappings in memory to reduce database query latency. In some embodiments, the resolution process may query multiple databases in sequence or parallel, such as first checking a local cache, then a primary database, and finally a backup or archival database if the mapping is not found in primary sources. The group identifier format may alternatively use different delimiters such as hyphens or periods, or may encode additional information such as facility codes or device categories within the identifier string. Connection parameters may alternatively be stored in separate configuration files referenced by pointers in the database, or may be dynamically generated based on device type templates and facility-specific network configurations.

At 306, the process queries the connection state manager to determine whether an active SignalR connection already exists for the target wearable device. The connection state manager maintains a registry of established connections between the server and various wearable devices, tracking connection identifiers, device identifiers, connection timestamps, and connection status indicators.

At 306, this process enables the server to avoid establishing redundant connections to wearable devices that are already connected, thereby conserving system resources and preventing connection conflicts. The process also facilitates connection reuse for subsequent communication requests directed to the same wearable device, reducing latency and improving overall system efficiency.

At 306, the process comprises receiving a query from the server containing the resolved device identifier obtained from previous processing steps. The connection state manager executes a lookup operation within its internal data structures, which may comprise hash tables, dictionaries, or database tables indexed by device identifiers. The lookup operation searches for entries matching the provided device identifier and examines the associated connection status fields. The connection state manager evaluates whether any matching entry indicates an active connection state, which may be determined by examining connection status flags, checking connection heartbeat timestamps against timeout thresholds, or verifying the underlying SignalR connection object remains valid. Upon completing the lookup and evaluation operations, the connection state manager returns a connection status response to the server, indicating either the presence of an active connection along with its connection details, or the absence of an active connection requiring establishment of a new connection.

At 306, alternative implementations may employ a distributed cache system, such as Redis or Memcached, to maintain connection state information across multiple server instances in a load-balanced environment. In such implementations, the process queries the distributed cache using the device identifier as a key, and the cache returns the connection state information if present. Another alternative utilizes a connection pool manager that maintains pre-established SignalR connections in a pool structure, where the server queries the pool manager to retrieve an available connection for the target device or requests allocation of a new connection from the pool. A further alternative implements a peer-to-peer discovery mechanism where the server broadcasts a connection status query to other server instances in a cluster, and peer servers respond with connection state information for the target device if they maintain such connections. Yet another alternative employs a centralized connection broker service that all servers query to determine connection states and coordinate connection establishment across the distributed system.

At 308, if no existing connection is found, the process creates a task completion source for the pending connection, then transmits a connection initiation request to the watch service server via SignalR. The watch service sends a notification to the target wearable device instructing it to establish a SignalR connection to the covert monitoring hub.

At 308, the software process addresses scenarios where the server determines that no active connection exists between the monitoring system and the target wearable device. This determination triggers a connection establishment sequence that enables real-time bidirectional communication between the server infrastructure and the wearable device through intermediary services.

At 308, the process operates by first instantiating a task completion source object that serves as an asynchronous placeholder for the pending connection operation. The task completion source provides a mechanism for tracking the connection establishment status and enables the server to await the completion of the connection process before proceeding with subsequent operations. Upon creating the task completion source, the process formulates a connection initiation request containing relevant parameters such as device identifiers, authentication tokens, connection parameters, and session information. This request is transmitted to the watch service server using the SignalR protocol, which provides real-time web functionality enabling server-to-client push communications. The watch service server, upon receiving the connection initiation request, processes the request parameters and generates a corresponding notification message formatted according to the wearable device’s communication specifications. The watch service server then transmits this notification to the target wearable device through established communication channels, which may include push notification services, persistent socket connections, or polling mechanisms. The notification instructs the wearable device to initiate a SignalR connection to the covert monitoring hub, providing connection endpoints, authentication credentials, and configuration parameters required for establishing the connection.

At 308, alternative implementations may employ different communication protocols and architectures while achieving the same functional outcome. Instead of SignalR, the system may utilize WebSocket connections directly, implementing custom handshake and message framing protocols to establish bidirectional communication channels. The connection initiation request may alternatively be transmitted using RESTful API calls over HTTPS, with the watch service server maintaining a message queue for pending connection requests. Rather than using a task completion source, the process may implement promise-based asynchronous patterns or callback mechanisms to handle the pending connection state. The notification to the wearable device may be delivered through alternative channels such as SMS-based triggers, Bluetooth Low Energy advertisements from nearby beacon devices, or through companion mobile applications that relay connection instructions. The system may also implement a polling-based approach where the wearable device periodically checks with the watch service server for pending connection requests, eliminating the need for push notifications. Additionally, the connection establishment may utilize MQTT protocol for lightweight publish-subscribe messaging, where the watch service server publishes connection requests to topics subscribed by target wearable devices.

At 310, the process comprises establishing a secure bidirectional connection between the wearable device and the SignalR hub. This connection establishment process enables real-time, persistent communication between the wearable device and the server infrastructure, facilitating continuous data exchange and remote command execution throughout the duration of the device’s operational session.

At 310, the connection establishment process provides a communication channel that supports both server-to-client and client-to-server messaging patterns. The bidirectional nature of the connection allows the wearable device to transmit sensor data, status updates, and event notifications to the server while simultaneously receiving configuration updates, control commands, and data requests from the server. The secure aspect of the connection ensures that all data transmitted between the wearable device and the server is protected against unauthorized access and tampering through encryption and authentication mechanisms.

At 310, the connection establishment operates through a multi-phase process. The wearable device initiates the connection by sending a connection request to the SignalR hub endpoint. Upon receiving the connection request, the SignalR hub authenticates the device using the provided credentials and establishes a WebSocket connection when supported by the network infrastructure. Once the underlying transport connection is established, the wearable device invokes the Register method on the hub, passing its group identifier as a parameter. The device registration engine receives this registration request and performs several operations: it validates the group identifier against a predefined set of valid groups, adds the device connection to the specified group within the hub’s group management system, signals a task completion source to indicate successful registration, and updates the connection state manager to reflect the device’s active status. The group membership enables the server to send targeted messages to specific sets of devices sharing common characteristics or operational requirements.

At 310, alternative implementations for establishing the secure bidirectional connection include utilizing gRPC streaming for bidirectional communication, where the wearable device opens a streaming RPC call that remains active for the duration of the session. Another alternative involves implementing a message queue-based architecture using protocols such as MQTT or AMQP, where the wearable device connects to a message broker and subscribes to relevant topics while publishing its data to designated queues. A further alternative employs WebRTC data channels, establishing peer-to-peer connections between the wearable device and the server with optional relay servers for NAT traversal. Additionally, the connection could be established using Server-Sent Events (SSE) combined with traditional HTTP POST requests, where SSE provides server-to-client communication while HTTP requests handle client-to-server messaging. For the registration process, alternatives include implementing a REST-based registration endpoint where the device posts its registration information and receives a connection token for subsequent WebSocket establishment, or utilizing a certificate-based mutual TLS authentication where device group membership is encoded within the client certificate’s subject alternative names.

At 312, the process may comprise a monitoring activation signal that specifies the particular type of monitoring data requested from the wearable device. The monitoring activation signal comprises a data structure containing parameters that identify whether audio data, screen capture data, or camera image data is to be collected from the target wearable device.

At 312, the process may execute a signal generation module that creates distinct activation signals based on the monitoring data type selected by an administrator or automated monitoring system. When audio monitoring is required, the server constructs a start audio monitor signal containing audio sampling parameters such as sample rate, bit depth, and encoding format. When screen capture monitoring is required, the server constructs a get screen capture signal containing display resolution parameters and image compression settings. When camera image monitoring is required, the server constructs a get camera image signal containing camera selection parameters, image resolution settings, and capture frequency indicators. Alternatively, a monitoring signal may be automatically generated by the user device based on certain environmental conditions around the user device, such as upon detecting certain sounds (e.g. loud noised), detecting certain keywords, detecting certain motion (e.g. fall detection), and the like.

At 312, the process implements the signal transmission through a SignalR hub infrastructure that maintains persistent WebSocket connections with the wearable devices. The SignalR hub operates as a real-time communication framework that enables bi-directional messaging between the server and connected wearable devices. Upon generating the appropriate monitoring activation signal, the server invokes the SignalR hub’s broadcast method to transmit the signal to the specified wearable device endpoint. The SignalR hub manages connection state, handles reconnection logic, and ensures reliable delivery of the monitoring activation signals through its underlying transport protocols.

At 312, alternative implementations may utilize different real-time communication protocols for transmitting the monitoring activation signals. The server may employ WebRTC (Web Real-Time Communication) protocols to establish peer-to-peer connections with the wearable devices for transmitting activation signals. The server may implement MQTT (Message Queuing Telemetry Transport) protocol with a message broker to publish monitoring activation signals to device-specific topics that the wearable devices subscribe to. The server may utilize gRPC (Google Remote Procedure Call) framework to invoke remote monitoring procedures on the wearable devices through strongly-typed service definitions. The server may implement custom TCP or UDP socket connections to transmit binary-encoded monitoring activation signals directly to the wearable devices’ network endpoints.

At 314, the process comprises receiving monitoring data from the wearable device via the established SignalR connection. The monitoring data comprises multiple data types transmitted through distinct channels within the SignalR framework, including audio data streams and visual capture data.

At 314, the process comprises processing incoming monitoring data streams from the wearable device, enabling real-time data acquisition and handling. The server maintains separate data reception pathways for different monitoring modalities, allowing concurrent processing of audio streams and visual captures. The received data enables the server to maintain a comprehensive monitoring state of the wearable device’s environment and user interactions.

At 314, the process implements specialized reception mechanisms for each data type. For audio monitoring, the process comprises receiving streaming audio packets through the audio stream method, which processes continuous audio data transmitted as discrete packets over the SignalR connection. The audio stream method handles packet sequencing, buffering, and reassembly to reconstruct the audio stream. For screen and camera captures, the process comprises receiving image data through respective acquisition modules. Each acquisition module processes incoming image data independently, with the screen acquisition module handling display capture data and the camera acquisition module handling camera feed data. Upon successful data receipt, task completion sources are signaled, providing asynchronous notification mechanisms that indicate when data reception operations have completed. These task completion sources enable downstream processing components to initiate subsequent operations upon confirmed data availability.

At 314, alternative implementations may utilize WebRTC (Web Real-Time Communication) protocols for receiving monitoring data, particularly for audio and video streams, providing peer-to-peer communication capabilities with built-in codec support. The server may alternatively implement WebSocket connections with custom protocol definitions for data transmission, offering lower-level control over the communication channel. For audio data, the process may receive data in alternative formats such as JPEG, WAV, MP3, or AAC encoded streams, with corresponding decoding modules processing the received packets. For visual captures, the process may receive PNG image data instead of JPEG, providing lossless compression for scenarios requiring higher image fidelity. The server may alternatively receive H.264 or H.265 encoded video streams for continuous visual monitoring rather than discrete image captures. The task completion sources may be replaced with callback mechanisms, event-driven notifications, or promise-based asynchronous patterns for signaling data receipt completion. The server may implement MQTT (Message Queuing Telemetry Transport) protocol for receiving monitoring data in bandwidth-constrained environments, with topic-based routing for different data types. Alternative implementations may employ gRPC streaming for bidirectional data flow, enabling server-to-client acknowledgments within the same connection context.

At 316, the process comprises transmitting the received monitoring data to the client computing device through the SignalR hub group connection. The transmission operation involves the server utilizing the established SignalR hub infrastructure to deliver various types of monitoring data to one or more connected client devices within a designated device group.

At 316, the process performs differentiated data transmission based on the type of monitoring data being processed. The server identifies the data type and applies corresponding transmission protocols to ensure proper delivery to the intended recipients. The transmission mechanism maintains real-time or near-real-time communication capabilities between the server and client devices.

At 316, the process operates by first receiving monitoring data from source devices or sensors. Upon receipt, the process examines the data type to determine the appropriate transmission method. For audio stream data, the process implements a broadcast mechanism wherein audio packets are replicated and transmitted to all client computing devices that maintain active connections within the specified SignalR hub group. This echoing process ensures that each connected client receives identical audio packet streams, enabling synchronized audio monitoring across multiple client devices. For image data, the process employs a request-response pattern where captured images are transmitted specifically to the client device that initiated the image capture request. The server maintains request tracking to ensure proper routing of image data to the corresponding requesting client. Additionally, the process generates and transmits connection status information to client devices, providing real-time feedback regarding the state of the communication channel and the availability of monitoring services.

At 316, alternative implementations may utilize WebSocket connections for direct bidirectional communication between the server and client devices. The server may implement long polling mechanisms where clients periodically request updates from the server, and the server holds the request open until new monitoring data becomes available. Server-Sent Events (SSE) may be employed for unidirectional data flow from the server to clients, particularly suitable for streaming monitoring data. The server may alternatively implement a message queuing system where monitoring data is placed in queues and clients retrieve messages asynchronously. For audio streams, the process may utilize WebRTC protocols to establish peer-to-peer connections between clients after initial server-mediated setup. The server may implement RESTful API endpoints where clients poll for new monitoring data at regular intervals. MQTT protocol may be employed for lightweight publish-subscribe messaging, where the server publishes monitoring data to topics and clients subscribe to receive updates. The server may utilize gRPC streaming for efficient binary data transmission with support for both unary and streaming communication patterns.

At 318, the process executes a monitoring process for active audio monitoring sessions by detecting periodic keep-alive signals transmitted from connected clients. The keep-alive signals comprise data packets sent by client devices to indicate their continued presence and active participation in the audio monitoring session. The server maintains a data structure storing session identifiers, client connection information, and timestamp data corresponding to the most recent keep-alive signal received from each connected client.

At 318, the monitoring process operates by implementing a timeout mechanism that tracks elapsed time since the last keep-alive signal reception for each active audio monitoring session. The server compares the current system time against the stored timestamp of the last received keep-alive signal for each client connection. When the elapsed time exceeds a predetermined timeout threshold value, the process identifies the corresponding audio monitoring session as inactive or disconnected. The timeout threshold value is configurable through server configuration parameters and may be adjusted based on network conditions, system requirements, or administrative preferences.

At 318, the start audio monitor process serves as the keep-alive signal generation mechanism on the client side. Client applications invoke this method at predetermined intervals to transmit keep-alive signals to the server. The method generates and transmits a data packet containing the client’s session identifier, timestamp information, and optionally, status data indicating the client’s current state. The server receives these periodic method calls through its network interface and updates the corresponding session’s timestamp in its monitoring data structure. Upon detecting absence of keep-alive signals within the timeout period, the process initiates a termination sequence that releases allocated resources associated with the inactive audio stream, including memory buffers, network sockets, and processing threads dedicated to that particular session.

At 318, alternative implementations of the keep-alive monitoring mechanism include utilizing WebSocket ping/pong frames where the server sends ping frames to clients and expects pong responses within the timeout period. Another alternative involves implementing a heartbeat protocol using UDP datagrams where clients send lightweight heartbeat packets at regular intervals, and the server tracks these packets to determine session activity. The monitoring process may alternatively employ TCP keep-alive options at the transport layer, where the operating system automatically sends keep-alive probes and notifies the application when connections become unresponsive. Additionally, the process may implement a hybrid approach combining application-level keep-alive signals with transport-layer mechanisms to provide redundant connection monitoring. The timeout period may be dynamically adjusted based on network latency measurements, with longer timeouts for high-latency connections and shorter timeouts for local network connections.

At 320, the process transmits the monitoring data to the data storage server for persistent archival. The monitoring data comprises multiple data types collected during operator sessions, including audio recordings captured from microphone inputs, screen captures obtained through display buffer sampling, camera images acquired from video input devices, and session metadata containing operational parameters and system states.

At 320, the transmission process packages the collected monitoring data with associated temporal and identification information. Each data element receives a timestamp indicating the precise time of capture, a device identifier specifying the source equipment or terminal from which the data originated, and operator information identifying the user associated with the monitored session. The server formats this aggregated data into a transmission-compatible structure for transfer to the data storage server.

At 320, the process establishes a communication channel with the data storage server using network protocols such as TCP/IP, HTTPS, or proprietary data transfer protocols. The monitoring data undergoes serialization into a transmittable format, which may include compression algorithms to reduce bandwidth requirements and encryption methods to secure the data during transit. The server initiates the data transfer by sending header information containing metadata about the transmission, followed by the payload containing the actual monitoring data. The data storage server receives the transmitted data, performs integrity verification through checksums or hash validation, and commits the data to persistent storage media. The storage operation preserves the hierarchical relationship between the monitoring data elements and their associated timestamps, device identifiers, and operator information, enabling subsequent retrieval based on various search criteria.

At 320, alternative transmission approaches include implementing a message queuing system where the server places monitoring data into a message queue, and the data storage server asynchronously retrieves and processes the queued messages. Another alternative involves utilizing a streaming data pipeline where the server continuously streams monitoring data to the data storage server through persistent connections, eliminating the overhead of establishing new connections for each transmission. The server may alternatively employ a batch transmission approach, accumulating monitoring data over predetermined intervals before transmitting consolidated data packages to reduce network overhead. In configurations with distributed storage architectures, the process may transmit monitoring data to multiple data storage servers simultaneously or in a round-robin fashion to distribute storage load and provide redundancy. The transmission process may alternatively incorporate data transformation operations, converting the monitoring data into standardized formats such as JSON, XML, or binary protocols before transmission to ensure compatibility with diverse storage systems.

In one aspect, storing monitoring data may comprise processing captured audio data through a speech-to-text transcription engine to generate text transcripts for storage. The speech-to-text transcription engine receives digital audio data as input and produces corresponding textual representations of the spoken content contained within the audio data. The transcription engine operates on audio data that has been captured from various sources, including but not limited to microphone inputs, telephony systems, video conference streams, or pre-recorded audio files. The speech-to-text transcription process converts human speech patterns encoded in the audio data into machine-readable text format. The transcription engine analyzes acoustic features of the audio signal, identifies phonemes and words, and constructs grammatically coherent text sequences that represent the original spoken content. The resulting text transcripts maintain temporal alignment with the source audio, enabling synchronization between the audio and text representations.

The speech recognition algorithms employed by the transcription engine utilize acoustic modeling, language modeling, and decoding techniques to perform the audio-to-text conversion. The acoustic model processes the audio signal to extract acoustic features such as mel-frequency cepstral coefficients (MFCCs) or filter bank energies. These features are analyzed to identify phonetic units within the audio stream. The language model applies statistical or neural network-based approaches to predict word sequences based on linguistic patterns and contextual information. The decoder combines outputs from the acoustic and language models to generate the most probable text sequence corresponding to the input audio. The transcription engine may employ deep neural networks, recurrent neural networks, transformer architectures, or hybrid approaches that combine multiple techniques. The engine processes audio data in segments, typically ranging from milliseconds to several seconds in duration, and maintains state information across segments to preserve context and improve transcription accuracy.

Alternative implementations for the speech-to-text transcription functionality include utilizing cloud-based speech recognition services accessed through application programming interfaces (APIs), employing on-premises speech recognition software libraries, or implementing distributed transcription systems where processing is divided between edge devices and centralized servers. The transcription engine may alternatively employ speaker diarization to identify and label different speakers within the audio stream, or utilize multi-language recognition capabilities to process audio containing multiple languages. Alternative acoustic modeling approaches include using Gaussian mixture models, deep belief networks, or convolutional neural networks. Alternative language modeling techniques include n-gram models, neural language models, or contextual embedding models. The system may alternatively implement real-time transcription with minimal latency for live audio streams, or batch processing for pre-recorded audio files where processing time is less constrained.

In one aspect, step 320's archival process further comprises an automated multi-stage analysis pipeline executed by the covert monitoring server. While streaming audio and video data are captured in parallel, the server bifurcates the incoming audio packet stream. The first branch is pushed to transcription engine 220, which converts the audio to text; this text is immediately consumed by keyword tracking engine 222, which applies string-matching algorithms to detect illicit communications. The second branch of the audio stream is pushed to voice biometrics engine 224, which dynamically validates the wearer's identity by correlating the stream's acoustic properties against the historical biometric hash vector on file.

Once the streaming session terminates or reaches a processing interval, inmate profile manager 226 takes the validated biometric metrics, any flagged keyword logs, the complete text transcript, and the raw H.264/H.265 video recordings, and packages them into an encrypted multi-media payload. The inmate profile manager 226 then executes an atomic database transaction on data storage server 102, appending this payload directly into the target user's master inmate profile, ensuring complete chain-of-custody data collection tied explicitly to the individual's institutional record.

### Hardware Architecture

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

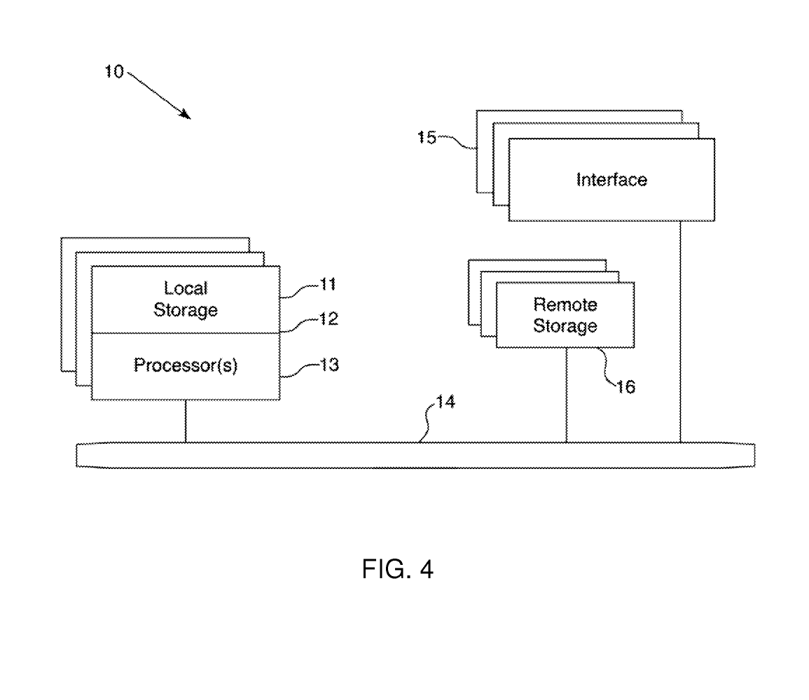

Software/hardware hybrid implementations of at least some of the embodiments disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific embodiments, at least some of the features or functionalities of the various embodiments disclosed herein may be implemented on one or more general-purpose computers associated with one or more networks, such as for example an end-user computer system, a client computer, a network server or other server system, a mobile computing device (e.g., tablet computing device, mobile phone, smartphone, laptop, or other appropriate computing device), a consumer electronic device, a music player, or any other suitable electronic device, router, switch, or other suitable device, or any combination thereof. In at least some embodiments, at least some of the features or functionalities of the various embodiments disclosed herein may be implemented in one or more virtualized computing environments (e.g., network computing clouds, virtual machines hosted on one or more physical computing machines, or other appropriate virtual environments). Any of the above mentioned systems, units, modules, engines, controllers, components, process steps or the like may be and/or comprise hardware and/or software as described herein. For example, the systems, engines, and subcomponents described herein may be and/or comprise computing hardware and/or software as described herein in association with FIGS. 4-7. Furthermore, any of the above mentioned systems, units, modules, engines, controllers, components, interfaces or the like may use and/or comprise an application programming interface (API) for communicating with other systems units, modules, engines, controllers, components, interfaces or the like for obtaining and/or providing data or information.

Referring now to FIG. 4, there is shown a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software- or hardware-based instructions according to one or more programs stored in memory. Computing device 10 may be configured to communicate with a plurality of other computing devices, such as clients or servers, over communications networks such as a wide area network a metropolitan area network, a local area network, a wireless network, the Internet, or any other network, using known protocols for such communication, whether wireless or wired.

In one aspect, computing device 10 includes one or more central processing units (CPU) 12, one or more interfaces 15, and one or more busses 14 (such as a peripheral component interconnect (PCI) bus). When acting under the control of appropriate software or firmware, CPU 12 may be responsible for implementing specific functions associated with the functions of a specifically configured computing device or machine. For example, in at least one aspect, a computing device 10 may be configured or designed to function as a server system utilizing CPU 12, local memory 11 and/or remote memory 16, and interface(s) 15. In at least one aspect, CPU 12 may be caused to perform one or more of the different types of functions and/or operations under the control of software modules or components, which for example, may include an operating system and any appropriate applications software, drivers, and the like.

CPU 12 may include one or more processors 13 such as, for example, a processor from one of the Intel, ARM, Qualcomm, and AMD families of microprocessors. In some embodiments, processors 13 may include specially designed hardware such as application-specific integrated circuits (ASICs), electrically erasable programmable read-only memories (EEPROMs), field-programmable gate arrays (FPGAs), and so forth, for controlling operations of computing device 10. In a particular aspect, a local memory 11 (such as non-volatile random-access memory (RAM) and/or read-only memory (ROM), including for example one or more levels of cached memory) may also form part of CPU 12. However, there are many different ways in which memory may be coupled to system 10. Memory 11 may be used for a variety of purposes such as, for example, caching and/or storing data, programming instructions, and the like. It should be further appreciated that CPU 12 may be one of a variety of system-on-a-chip (SOC) type hardware that may include additional hardware such as memory or graphics processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may for example support other peripherals used with computing device 10. Among the interfaces that may be provided are Ethernet interfaces, frame relay interfaces, cable interfaces, DSL interfaces, token ring interfaces, graphics interfaces, and the like. In addition, various types of interfaces may be provided such as, for example, universal serial bus (USB), Serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast Ethernet interfaces, Gigabit Ethernet interfaces, Serial ATA (SATA) or external SATA (ESATA) interfaces, high-definition multimedia interface (HDMI), digital visual interface (DVI), analog or digital audio interfaces, asynchronous transfer mode (ATM) interfaces, high-speed serial interface (HSSI) interfaces, Point of Sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 4 illustrates one specific architecture for a computing device 10 for implementing one or more of the embodiments described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, single processor 13 handles communications as well as routing computations, while in other embodiments a separate dedicated communications processor may be provided. In various embodiments, different types of features or functionalities may be implemented in a system according to the aspect that includes a client device (such as a tablet device or smartphone running client software) and server systems (such as a server system described in more detail below).

Regardless of network device configuration, the system of an aspect may employ one or more memories or memory modules (such as, for example, remote memory block 16 and local memory 11) configured to store data, program instructions for the general-purpose network operations, or other information relating to the functionality of the embodiments described herein (or any combinations of the above). Program instructions may control execution of or comprise an operating system and/or one or more applications, for example. Memory 16 or memories 11, 16 may also be configured to store data structures, configuration data, encryption data, historical system operations information, or any other specific or generic non-program information described herein.

Because such information and program instructions may be employed to implement one or more systems or methods described herein, at least some network device embodiments may include nontransitory machine-readable storage media, which, for example, may be configured or designed to store program instructions, state information, and the like for performing various operations described herein. Examples of such nontransitory machine- readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device), or they may be removable such as swappable flash memory modules (such as “thumb drives” or other removable media designed for rapidly exchanging physical storage devices), “hot-swappable” hard disk drives or solid state drives, removable optical storage discs, or other such removable media, and that such integral and removable storage media may be utilized interchangeably. Examples of program instructions include both object code, such as may be produced by a compiler, machine code, such as may be produced by an assembler or a linker, byte code, such as may be generated by for example a JAVA™ compiler and may be executed using a Java virtual machine or equivalent, or files containing higher level code that may be executed by the computer using an interpreter (for example, scripts written in Python, Perl, Ruby, Groovy, or any other scripting language).

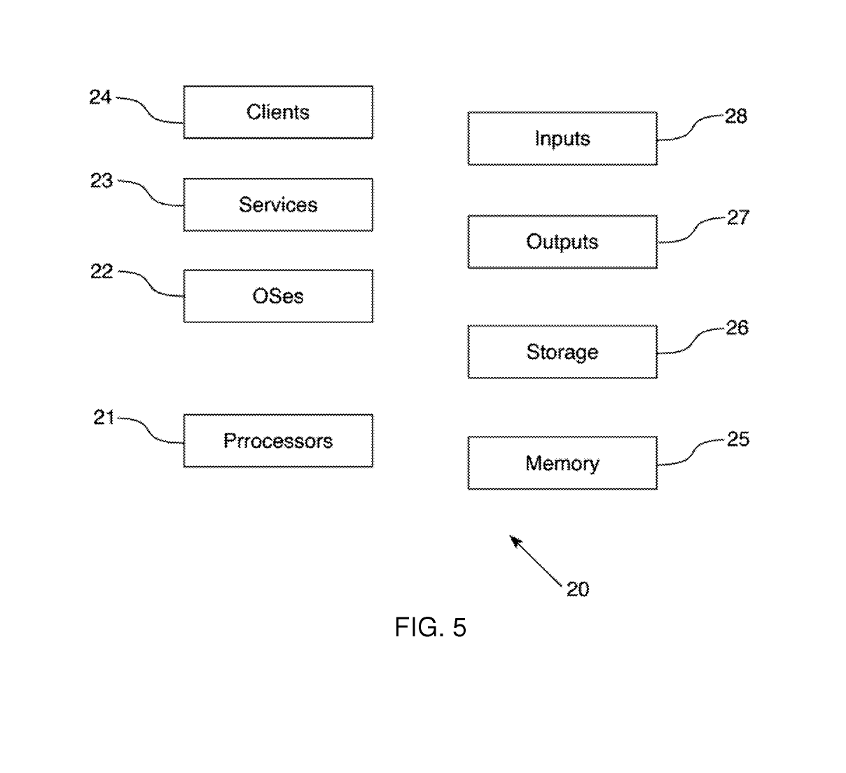

In some embodiments, systems may be implemented on a standalone computing system. Referring now to FIG. 5, there is shown a block diagram depicting a typical exemplary architecture of one or more embodiments or components thereof on a standalone computing system. Computing device 20 includes processors 21 that may run software that carry out one or more functions or applications of embodiments, such as for example a client application. Processors 21 may carry out computing instructions under control of an operating system 22 such as, for example, a version of MICROSOFT WINDOWS™ operating system, APPLE macOS™ or iOS™ operating systems, some variety of the Linux operating system, ANDROID™ operating system, or the like. In many cases, one or more shared services 23 may be operable in system 20, and may be useful for providing common services to client applications. Services 23 may for example be WINDOWS™ services, user-space common services in a Linux environment, or any other type of common service architecture used with operating system 21. Input devices 28 may be of any type suitable for receiving user input, including for example a keyboard, touchscreen, microphone (for example, for voice input), mouse, touchpad, trackball, or any combination thereof. Output devices 27 may be of any type suitable for providing output to one or more users, whether remote or local to system 20, and may include for example one or more screens for visual output, speakers, printers, or any combination thereof. Memory 25 may be random-access memory having any structure and architecture known in the art, for use by processors 21, for example to run software. Storage devices 26 may be any magnetic, optical, mechanical, memristor, or electrical storage device for storage of data in digital form (such as those described above, referring to FIG. 4). Examples of storage devices 26 include flash memory, magnetic hard drive, CD-ROM, and/or the like.

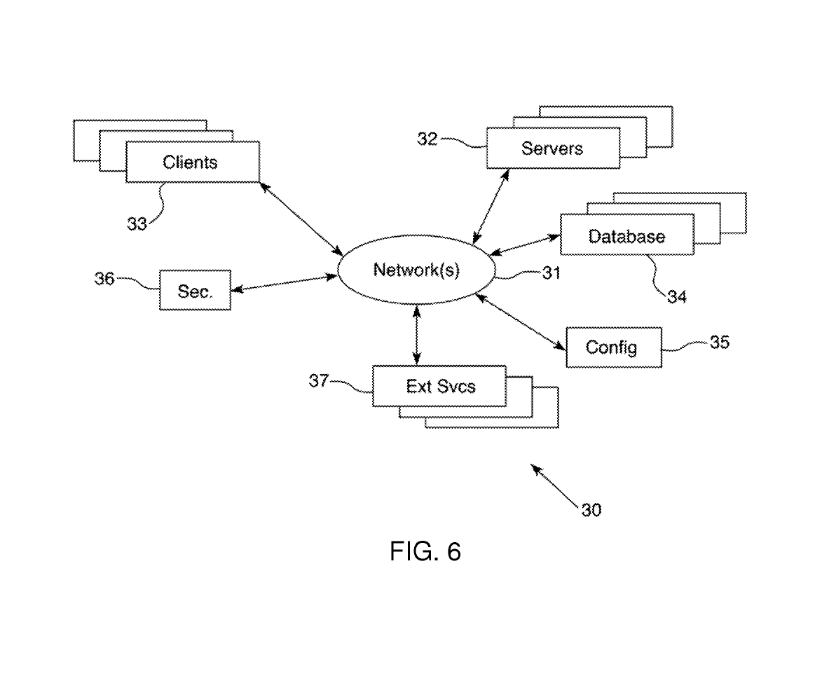

In some embodiments, systems may be implemented on a distributed computing network, such as one having any number of clients and/or servers. Referring now to FIG. 6, there is shown a block diagram depicting an exemplary architecture 30 for implementing at least a portion of a system according to one aspect on a distributed computing network. According to the aspect, any number of clients 33 may be provided. Each client 33 may run software for implementing client-side portions of a system; clients may comprise a system 20 such as that illustrated in FIG. 5. In addition, any number of servers 32 may be provided for handling requests received from one or more clients 33. Clients 33 and servers 32 may communicate with one another via one or more electronic networks 31, which may be in various embodiments any of the Internet, a wide area network, a mobile telephony network (such as CDMA or GSM cellular networks), a wireless network (such as WiFi, WiMAX, LTE, and so forth), or a local area network (or indeed any network topology known in the art; the aspect does not prefer any one network topology over any other). Networks 31 may be implemented using any known network protocols, including for example wired and/or wireless protocols.

In addition, in some embodiments, servers 32 may call external services 37 when needed to obtain additional information, or to refer to additional data concerning a particular call. Communications with external services 37 may take place, for example, via one or more networks 31. In various embodiments, external services 37 may comprise web-enabled services or functionality related to or installed on the hardware device itself. For example, in one aspect where client applications are implemented on a smartphone or other electronic device, client applications may obtain information stored in a server system 32 in the cloud or on an external service 37 deployed on one or more of a particular enterprise’s or user’s premises.

In some embodiments, clients 33 or servers 32 (or both) may make use of one or more specialized services or appliances that may be deployed locally or remotely across one or more networks 31. For example, one or more databases 34 may be used or referred to by one or more embodiments. It should be understood by one having ordinary skill in the art that databases 34 may be arranged in a wide variety of architectures and using a wide variety of data access and manipulation means. For example, in various embodiments one or more databases 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some embodiments, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system, or a logical database within an overall database management system. Unless a specific meaning is specified for a given use of the term “database”, it should be construed to mean any of these senses of the word, all of which are understood as a plain meaning of the term “database” by those having ordinary skill in the art.

Similarly, some embodiments may make use of one or more security systems 36 and configuration systems 35. Security and configuration management are common information technology (IT) and web functions, and some amount of each are generally associated with any IT or web systems. It should be understood by one having ordinary skill in the art that any configuration or security subsystems known in the art now or in the future may be used in conjunction with embodiments without limitation, unless a specific security 36 or configuration system 35 or approach is specifically required by the description of any specific aspect.

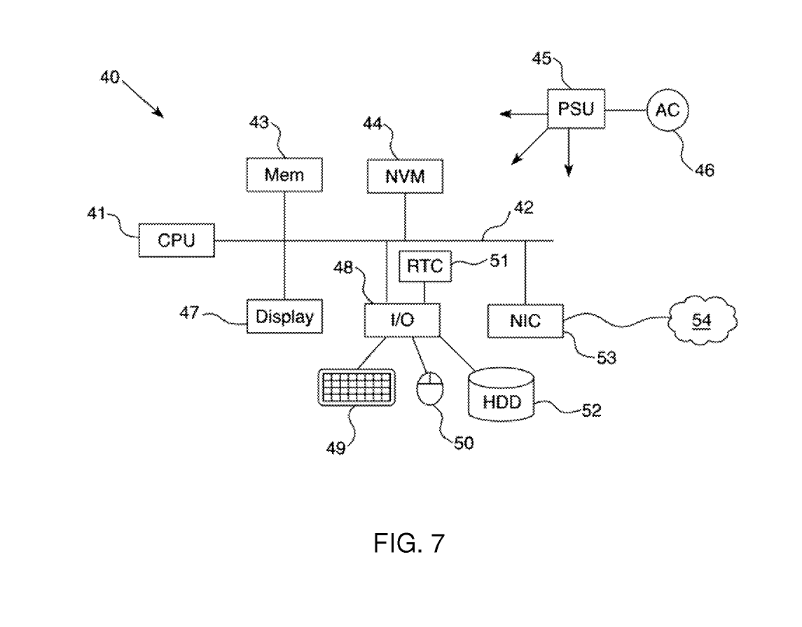

FIG. 7 shows an exemplary overview of a computer system 40 as may be used in any of the various locations throughout the system. It is exemplary of any computer that may execute code to process data. Various modifications and changes may be made to computer system 40 without departing from the broader scope of the system and method disclosed herein. Central processor unit (CPU) 41 is connected to bus 42, to which bus is also connected memory 43, nonvolatile memory 44, display 47, input/output (I/O) unit 48, and network interface card (NIC) 53. I/O unit 48 may, typically, be connected to keyboard 49, pointing device 50, hard disk 52, and real-time clock 51. NIC 53 connects to network 54, which may be the Internet or a local network, which local network may or may not have connections to the Internet. Also shown as part of system 40 is power supply unit 45 connected, in this example, to a main alternating current (AC) supply 46. Not shown are batteries that could be present, and many other devices and modifications that are well known but are not applicable to the specific novel functions of the current system and method disclosed herein. It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles, or other integrated hardware devices).

In various embodiments, functionality for implementing systems or methods of various embodiments may be distributed among any number of client and/or server components. For example, various software modules may be implemented for performing various functions in connection with the system of any particular aspect, and such modules may be variously implemented to run on server and/or client components.

The skilled person will be aware of a range of possible modifications of the various embodiments described above. Accordingly, the present invention is defined by the claims and their equivalents.

### Additional Considerations

As used herein any reference to "one embodiment" or "an embodiment" means that a particular element, feature, structure, or characteristic described in connection with the embodiment is included in at least one embodiment. The appearances of the phrase "in one embodiment" in various places in the specification are not necessarily all referring to the same embodiment.

Some embodiments may be described using the expression "coupled" and "connected" along with their derivatives. For example, some embodiments may be described using the term "coupled" to indicate that two or more elements are in direct physical or electrical contact. The term "coupled," however, may also mean that two or more elements are not in direct contact with each other, but yet still co-operate or interact with each other. The embodiments are not limited in this context.

As used herein, the terms "comprises," "comprising," "includes," "including," "has," "having" or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, "or" refers to an inclusive or and not to an exclusive or. For example, a condition A or B is satisfied by any one of the following: A is true (or present) and Bis false (or not present), A is false ( or not present) and Bis true ( or present), and both A and B are true ( or present).

In addition, use of the "a" or "an" are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and/or a process associated with the disclosed principles herein. Thus, while particular embodiments and applications have been illustrated and described, it is to be understood that the disclosed embodiments are not limited to the precise construction and components disclosed herein. Various apparent modifications,

changes and variations may be made in the arrangement, operation and details of the method and apparatus disclosed herein without departing from the spirit and scope defined in the appended claims.

What is claimed is:

1. A system for remotely monitoring audio and visual data from wearable computing devices, comprising:
a monitoring server comprising a processor and memory storing executable instructions that, when executed by the processor, cause the monitoring server to:
receive, via an application programming interface, a monitoring request containing a user identifier from a client computing device;
query a device database to resolve the user identifier to a specific wearable device identifier and associated connection parameters;
determine, via a connection state manager, whether an active bidirectional communication channel exists between the monitoring server and a target wearable device;
responsive to determining no active channel exists, generate a task completion source object and transmit a connection initiation signal to a watch service server, wherein the watch service server pushes a notification to the target wearable device instructing establishment of a secure connection;
establish a secure bidirectional connection with the target wearable device upon receiving a registration request containing a group identifier;
transmit monitoring activation signals to the target wearable device specifying requested data types selected from audio stream capture, screen capture, and/or camera image capture;
receive monitoring data packets from the target wearable device via the secure connection;
relay the received monitoring data to the client computing device through group-based connections; and
maintain active audio streaming sessions by monitoring for periodic keep-alive signals from the client computing device at predetermined intervals, wherein absence of a keep-alive signal within a timeout threshold triggers automatic stream termination.

2. The system of claim 1, wherein the executable instructions further cause the monitoring server to:
store the received monitoring data to a data storage server with associated metadata including timestamps, device identifiers, and operator information; and
process audio data through a speech-to-text transcription engine implementing speech recognition algorithms to generate searchable text transcripts.

3. The system of claim 1, wherein establishing the secure bidirectional connection comprises:
processing a device registration request at a device registration engine;
adding the target wearable device to a group corresponding to the group identifier;
signaling the task completion source object to indicate successful connection establishment; and
updating the connection state manager with current connection status.

4. The system of claim 1, wherein the monitoring activation signals comprise:
an audio capture signal for initiating audio stream capture;
a screen capture signal for requesting screen image data; and
a camera image capture signal for requesting camera image data.

5. The system of claim 1, wherein the executable instructions further cause the monitoring server to:
support multiple simultaneous client connections to a single wearable device through group-based connection management; and
echo audio stream packets to all connected clients within the device group.

6. A computer-implemented method for remotely monitoring audio and visual data from wearable computing devices, comprising:
receiving, by a monitoring server processor via an application programming interface, a monitoring request containing a user identifier from a client computing device;
automatically querying a device database to resolve the user identifier to a wearable device identifier and connection parameters;
determining, via a connection state manager, whether an active connection exists for the identified wearable device;
responsive to no existing connection, creating a task completion source and transmitting a connection initiation request to a watch service server, wherein the watch service server pushes a notification to the wearable device;
establishing a secure bidirectional connection upon receiving a device registration request containing a group identifier;
generating and transmitting monitoring activation signals specifying requested monitoring data types;
receiving monitoring data from the wearable device, wherein audio data is received as streaming packets and image data is received as JPEG-encoded data;
transmitting the monitoring data to the client computing device through group-based connections; and
maintaining audio streams by monitoring for periodic keep-alive signals, wherein stream termination occurs upon timeout.

7. The method of claim 6, further comprising:
storing monitoring data with metadata to a data storage server; and
processing audio data through speech recognition algorithms to generate text transcripts.

8. The method of claim 6, wherein establishing the connection comprises:
processing the registration request at a device registration engine;
adding the wearable device to a group;
signaling the task completion source; and
updating connection state in the connection state manager.

9. The method of claim 6, wherein the monitoring activation signals comprise distinct signal types for audio streaming, screen capture, and camera image capture.

10. The method of claim 6, further comprising supporting multiple simultaneous client connections to a single wearable device through group-based connection management.

11. A non-transitory computer-readable storage medium storing instructions that, when executed by a processor, cause the processor to perform operations for remotely monitoring wearable computing devices, the operations comprising:
receiving a monitoring request with a user identifier via an application programming interface;
resolving the user identifier to a wearable device identifier through database query;
determining active connection status via a connection state manager;
establishing a connection through task completion source generation and watch service notification when no connection exists;
transmitting monitoring activation signals for audio, screen, or camera data capture;
receiving streaming audio packets or image data from the wearable device;
relaying monitoring data to client devices through group connections; and
maintaining audio streams through keep-alive signal monitoring with automatic timeout termination.

12. The storage medium of claim 11, wherein the operations further comprise:
archiving monitoring data with metadata; and
converting audio to searchable text via speech recognition.

13. The storage medium of claim 11, wherein connection establishment comprises:
device registration processing;
group assignment;
task completion signaling; and
connection state updates.

14. The storage medium of claim 11, wherein the operations support multiple client connections per wearable device.

15. The storage medium of claim 11, wherein monitoring activation signals are specific to requested data types including audio capture, screen capture, and camera image capture signals.
